SEQUENCE LISTING (35 sequences)

SEQ ID NO: 1 - protein, 241 aa, Aequorea victoria
MSRVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 2 - DNA, 1150 bp, Aequorea victoria
  source 1..1150: mol_type = genomic DNA
atgtctagagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggaggtagatttatgcatcctcttgtcatgagaagtcgaattgttcccattctgtgtgttgcagctacagatggagatacatagagatactcgtggattttgcttagtgttgagttttgttctggttgtgaactaaaagtttatacatttgcaggaaataaatagccttttgtttaaatcaaaaggtcttacctatgttagtgtgaagcattggatcccaaagaactccaaaatgcgatgaggcatatttaatcttgtctggactagtaacaggttgggatgaccacctgtgaagctccaacaggattgcctcctcacgcaatgtttgaggtctgatgttcaatagcttgttttgtttcactttgctttggactttcttttcgccaatgagctatgtttctgatggttttcactcttttggtgtgtagagaaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 3 - protein, 239 aa, Aequorea victoria
MVSKGEELFTGVVPILVELDGDVNGHKFSVRGEGEGDATNGKLTLKFICTTGKLPVPWPTLVTTFGYGVACFSRYPDHMKQHDFFKSAMPEGYVQERTISFKDDGTYKTRAEVKFEGDTLVNRIELKGIDFKEDGNILGHKLEYNFNSHYVYITADKQKNCIKANFKIRHNVEDGSVQLADHYQQNTPIGDGPVLLPDNHYLSHQSKLSKDPNEKRDHMVLLEFVTAAGITHGMDELYK

SEQ ID NO: 4 - DNA, 717 bp, Aequorea victoria
  source 1..717: mol_type = genomic DNA
atggtgagcaagggcgaggagctgttcaccggggtggtgcccatcctggtcgagctggacggcgacgtaaacggccacaagttcagcgtccgcggcgagggcgagggcgatgccaccaacggcaagctgaccctgaagttcatctgcaccaccggcaagctgcccgtgccctggcccaccctcgtgaccaccttcggctacggcgtggcctgcttcagccgctaccccgaccacatgaagcagcacgacttcttcaagtccgccatgcccgaaggctacgtccaggagcgcaccatctctttcaaggacgacggtacctacaagacccgcgccgaggtgaagttcgagggcgacaccctggtgaaccgcatcgagctgaagggcatcgacttcaaggaggacggcaacatcctggggcacaagctggagtacaacttcaacagccactacgtctatatcacggccgacaagcagaagaactgcatcaaggctaacttcaagatccgccacaacgttgaggacggcagcgtgcagctcgccgaccactaccagcagaacacccccatcggcgacggccccgtgctgctgcccgacaaccactacctgagccatcagtccaagctgagcaaagaccccaacgagaagcgcgatcacatggtcctgctggagttcgtgaccgccgccgggattacacatggcatggacgagctgtacaag

SEQ ID NO: 5 - protein, 225 aa, Discosoma sp.
MDSTENVIKPFMRFKVHMEGSVNGHEFEIEGEGEGKPYEGTQTAKLQVTKGGPLPFAWDILSPQFQYGSKVYVKHPADIPDYKKLSFPEGFKWERVMNFEDGGVVTVTQDSSLQDGTFIYHVKFIGVNFPSDGPVMQKKTLGWEPSTERLYPRDGVLKGEIHKALKLKGGGHYLVEFKSIYMAKKPVKLPGYYYVDSKLDITSHNEDYTVVEQYERAEARHHLFQ

SEQ ID NO: 6 - DNA, 897 bp, Discosoma sp.
  source 1..897: mol_type = genomic DNA
aagctttcgaaaagccctgacaaccctcgttcctaaaaaggaataagcgtttggtcagtaaataatagaaataaaaaatcagacctaagactgatgacaaaaagagcaaattttgataaaatagtattaattactagttaaggaggacaaacatggatagcactgagaacgtcatcaagcccttcatgcgcttcaaggtgcacatggagggctccgtgaacggccacgagttcgagatcgagggcgagggcgagggcaagccctacgagggcacccagaccgccaagctgcaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggcaccttcatctaccacgtgaagttcatcggcgtgaacttcccctccgacggccccgtaatgcagaagaagactctgggctgggagccctccaccgagcgcctgtacccccgcgacggcgtgctgaagggcgagatccacaaggcgctgaagctgaagggcggcggccactacctggtggagttcaagtcaatctacatggccaagaagcccgtgaagctgcccggctactactacgtggactccaagctggacatcacctcccacaacgaggactacaccgtggtggagcagtacgagcgcgccgaggcccgccaccacctgttccagtagctcggtaccaaattccagaaaagaggcctcccgaaaggggggccttttttcgttttggtccgaattc

SEQ ID NO: 7 - protein, 236 aa, Discosoma sp.
MVSKGEENNMAIIKEFMRFKVHMEGSVNGHEFEIEGEGEGRPYEAFQTAKLKVTKGGPLPFAWDILSPQFMYGSKVYIKHPADIPDYFKLSFPEGFRWERVMNFEDGGIIHVNQDSSLQDGVFIYKVKLRGTNFPSDGPVMQKKTMGWEASEERMYPEDGALKSEIKKRLKLKDGGHYAAEVKTTYKAKKPVQLPGAYIVDIKLDIVSHNEDYTIVEQYERAEGRHSTGGMDELYK

SEQ ID NO: 8 - DNA, 711 bp, Discosoma sp.
  source 1..711: mol_type = genomic DNA
atggtgagcaagggcgaggagaataacatggccatcatcaaggagttcatgcgcttcaaggtgcacatggagggctccgtgaacggccacgagttcgagatcgagggcgagggcgagggccgcccctacgaggcctttcagaccgctaagctgaaggtgaccaagggtggccccctgcccttcgcctgggacatcctgtcccctcagttcatgtacggctccaaggtctacattaagcacccagccgacatccccgactacttcaagctgtccttccccgagggcttcaggtgggagcgcgtgatgaacttcgaggacggcggcattattcacgttaaccaggactcctccctgcaggacggcgtgttcatctacaaggtgaagctgcgcggcaccaacttcccctccgacggccccgtaatgcagaagaagaccatgggctgggaggcctccgaggagcggatgtaccccgaggacggcgccctgaagagcgagatcaagaagaggctgaagctgaaggacggcggccactacgccgccgaggtcaagaccacctacaaggccaagaagcccgtgcagctgcccggcgcctacatcgtcgacatcaagttggacatcgtgtcccacaacgaggactacaccatcgtggaacagtacgaacgcgccgagggccgccactccaccggcggcatggacgagctgtacaagtaa

SEQ ID NO: 9 - protein, 232 aa, synthetic construct
  REGION 1..232: note = Synthetic construct
MVSKGEAVIKEFMRFKVHMEGSMNGHEFEIEGEGEGRPYEGTQTAKLKVTKGGPLPFSWDILSPQFMYGSRAFIKHPADIPDYYKQSFPEGFKWERVMNFEDGGAVTVTQDTSLEDGTLIYKVKLRGTNFPPDGPVMQKKTMGWEASTERLYPEDGVLKGDIKMALRLKDGGRYLADFKTTYKAKKPVQMPGAYNVDRKLDITSHNEDYTVVEQYERSEGRHSTGGMDELYK

SEQ ID NO: 10 - DNA, 696 bp, synthetic construct
  misc_feature 1..696: note = Synthetic construct
  source 1..696: mol_type = other DNA
atggtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcatcaagcaccccgccgacatccccgactactataagcagtccttccccgagggcttcaagtgggagcgcgtgatgaacttcgaggacggcggcgccgtgaccgtgacccaggacacctccctggaggacggcaccctgatctacaaggtgaagctccgcggcaccaacttccctcctgacggccccgtaatgcagaagaagacaatgggctgggaagcgtccaccgagcggttgtaccccgaggacggcgtgctgaagggcgacattaagatggccctgcgcctgaaggacggcggccgctacctggcggacttcaagaccacctacaaggccaagaagcccgtgcagatgcccggcgcctacaacgtcgaccgcaagttggacatcacctcccacaacgaggactacaccgtggtggaacagtacgaacgctccgagggccgccactccaccggcggcatggacgagctgtacaag

SEQ ID NO: 11 - protein, 239 aa, Aequorea victoria
MVSKGEELFTGVVPILVELDGDVNGHKFSVRGEGEGDATNGKLTLKFICTTGKLPVPWPTLVTTLSHGVQCFARYPDHMKQHDFFKSAMPEGYVQERTIFFKDDGTYKTRAEVKFEGDTLVNRIELKGVDFKEDGNILGHKLEYNFNSHNIYIMAVKQKNGIKVNFKIRHNVEDGSVQLADHYQQNTPIGDGPVLLPDSHYLSTQSVLSKDPNEKRDHMVLLEFRTAAGITLGMDELYK

SEQ ID NO: 12 - DNA, 720 bp, Aequorea victoria
  source 1..720: mol_type = genomic DNA
atggtgagcaagggcgaggagctgttcaccggggtggtgcccatcctggtcgagctggacggcgacgtaaacggccacaagttcagcgtgaggggcgagggcgagggcgatgccaccaacggcaagctgaccctgaagttcatctgcaccaccggcaagctgcccgtgccctggcccaccctcgtgaccaccctgagccacggcgtgcagtgcttcgcccgctaccccgaccacatgaagcagcacgacttcttcaagtccgccatgcccgaaggctacgtccaggagcgcaccatcttcttcaaggacgacggcacctacaagacccgcgccgaggtgaagttcgagggcgacaccctggtgaaccgcatcgagctgaagggcgtcgacttcaaggaggacggcaacatcctggggcacaagctggagtacaacttcaacagccacaacatctatatcatggccgtcaagcagaagaacggcatcaaggtgaacttcaagatccgccacaacgtggaggacggcagcgtgcagctcgccgaccactaccagcagaacacccccatcggcgacggccccgtgctgctgcccgacagccactacctgagcacccagtccgtgctgagcaaagaccccaacgagaagcgcgatcacatggtcctgctggagttccgcaccgccgccgggatcactctcggcatggacgagctgtacaagtaa

SEQ ID NO: 13 - protein, 233 aa, Discosoma sp.
MSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANAKTTYRSKKPAKNLKMPGVYYVDYRLERIKEANNETYVEQHEVAVARYCDLPSKLGHKLN

SEQ ID NO: 14 - DNA, 699 bp, Discosoma sp.
  source 1..699: mol_type = genomic DNA
atgagcgagctgatcaaggagaacatgcacatgaagctgtacatggagggcaccgtggacaaccaccacttcaagtgcaccagcgagggcgagggcaagccgtacgagggcacccagaccatgcgcatcaaggtggtggagggcggcccgctgccgttcgccttcgacatcctggccaccagcttcctgtacggcagcaagaccttcatcaaccacacccagggcatcccggacttcttcaagcagagcttcccggagggcttcacctgggagcgcgtgaccacctacgaggacggcggcgtgctgaccgccacccaggacaccagcctgcaggacggctgcctgatctacaacgtgaagatccgcggcgtgaacttcaccagcaacggcccggtgatgcagaagaagaccctgggctgggaggccttcaccgaaaccctgtacccggccgacggcggcctggagggccgcaacgacatggccctgaagctggtgggcggcagccacctgatcgccaacgccaagaccacctaccgcagcaagaagccggccaagaacctgaagatgccgggcgtgtactacgtggactaccgcctggagcgcatcaaggaggccaacaacgaaacctacgtggagcagcacgaggtggccgtggcccgctactgcgacctgccgagcaagctgggccacaagctgaac

SEQ ID NO: 15 - protein, 239 aa, Aequorea victoria
MVSKGEELFTGVVPILVELDGDVNGHKFSVSGEGEGDATYGKLTLKFICTTGKLPVPWPTLVTTFGYGLMCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSYQSKLSKDPNEKRDHMVLLEFVTAAGITLGMDELYK

SEQ ID NO: 16 - DNA, 714 bp, Aequorea victoria
  source 1..714: mol_type = genomic DNA
gtatcgaagggagaggagttgtttacgggcgtcgtgccaattcttgtggagttggacggtgacgtgaatggtcacaaattctcggtcagcggggagggagaaggagatgccacttacggaaagctgacattgaagttcatctgtaccacgggtaaactccccgtaccgtggcctacgctcgtgacaacgttcggttacggccttatgtgtttcgcaagatatccggatcacatgaaacagcacgactttttcaagtccgccatgccggagggttacgtacaagagagaacgatctttttcaaagatgacggcaactacaagactcgcgcggaagtgaagtttgagggagatacccttgtgaatagaattgagcttaaggggatcgacttcaaagaggacggtaacattctgggccacaaacttgaatacaactataactcgcataatgtgtacattatggcggataaacagaagaatgggatcaaagtgaatttcaagatcaggcataacatcgaggacggatcagtgcagttggctgaccattatcagcagaacacgcccatcggtgacggtccggtgctgcttccggataaccactacctttcatatcagtcgaagctgtccaaagatcccaacgaaaagcgcgatcatatggtcctgctggagtttgtcaccgcggcaggaatcacattgggaatggacgaattgtataag

SEQ ID NO: 17 - protein, 239 aa, Aequorea victoria
MVSKGEELFTGVVPILVELDGDVNGHKFSVSGEGEGDATYGKLTLKFICTTGKLPVPWPTLVTTLTWGVQCFARYPDHMKQHDFFKSAMPEGYVQERTIFFKDDGNYKTRAEVKFEGDTLVNRIELKGIDFKEDGNILGHKLEYNAISDNVYITADKQKNGIKANFKIRHNIEDGSVQLADHYQQNTPIGDGPVLLPDNHYLSTQSALSKDPNEKRDHMVLLEFVTAAGITLGMDELYK

SEQ ID NO: 18 - DNA, 720 bp, Aequorea victoria
  source 1..720: mol_type = genomic DNA
atggtgtcaaaaggagaggaactgtttactggagtcgtgcctatcctggtggagctggatggagatgtgaatggacacaagttctctgtgtctggagagggagagggagatgctacatatggaaagctgacactgaagttcatctgtacaacaggaaagctgcctgtgccttggcctacactggtgacaacactgacatggggagtgcagtgttttgctagataccctgaccacatgaagcagcatgacttcttcaagtctgctatgcctgagggatatgtgcaggagagaacaatcttcttcaaggatgatggaaactacaagacaagagctgaggtgaagtttgagggagacacactggtgaacagaattgagctgaagggaattgacttcaaggaggatggaaacatcctgggacacaagctggagtacaatgctatctctgacaatgtgtacatcacagctgacaagcagaagaatggaatcaaggctaacttcaagatcagacacaacattgaggatggatctgtgcagctggctgaccactaccagcagaacacacctattggagatggacctgtgctgctgcctgacaaccactacctgagcacacagtctgctctgagcaaggaccctaatgagaagagagaccacatggtgctgctggagtttgtgacagctgctggaatcacactgggaatggatgagctgtacaagtaa

SEQ ID NO: 19 - protein, 240 aa, Discosoma sp.
MRQSSRGRMVSELIKENMHMKLYMEGTVNNHHFKCTSEGEGKPYEGTQTMRIKAVEGGPLPFAFDILATSFMYGSKTFINHTQGIPDFFKQSFPEGFTWERVTTYEDGGVLTATQDTSLQDGCLIYNVKIRGVNFPSNGPVMQKKTLGWEASTETLYPADGGLEGRADMALKLVGGGHLICNLKTTYRSKKPAKNLKMPGVYYVDRRLERIKEADKETYVEQHEVAVARYCDLPSKLGHR

SEQ ID NO: 20 - DNA, 696 bp, Discosoma sp.
  source 1..696: mol_type = genomic DNA
atggtgagcgagctgattaaggagaacatgcccatgaagctgtacatggagggcaccgtgaacaaccaccacttcaagtgcacatccgagggcgaaggcaagccctacgagggcacccagaccatgagaatcaaggtcgtcgagggcggccctctccccttcgccttcgacatcctggctaccagcttcatgtacggcagcagaaccttcatcaagcaccctccgggcatccccgacttctttaagcagtccttccctgagggcttcacatgggagagagtcaccacatacgaagacgggggcgtgctgaccgctacccaggacaccagcctccaggacggctgcctcatctacaacgtcaaggttagaggggtgaacttcccagccaacggccctgtgatgcagaagaaaacactcggctgggaggccgccaccgagacgatgtaccccgctgacggcggcctggaaggcgcagctgacatggccctgaagctcgtgggcgggggccacctgatctgcaaccttgagaccacatacagatccaagaaacccgctacgaacctcaagatgcccggcgtctacaacgtggaccacagactggaaagaatcaaggaggccgacgatgagacctacgtcgagcagcacgaggtggctgtggccagatactctactggtggcgctggtgatggaggtaaa

SEQ ID NO: 21 - protein, 550 aa, Discosoma sp.
MEDAKNIKKGPAPFYPLEDGTAGEQLHKAMKRYALVPGTIAFTDAHIEVNITYAEYFEMSVRLAEAMKRYGLNTNHRIVVCSENSLQFFMPVLGALFIGVAVAPANDIYNERELLNSMNISQPTVVFVSKKGLQKILNVQKKLPIIQKIIIMDSKTDYQGFQSMYTFVTSHLPPGFNEYDFVPESFDRDKTIALIMNSSGSTGSPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNDPEATNALIDKDGWLHSGDIAYWDEDEHFFIVDRLKSLIKYKGCQVAPAELESILLQHPNIFDAGVAGLPGDDAGELPAAVVVLEHGKTMTEKEIVDYVASQVTTAKKLRGGVVFVDEVPKGLTGKLDARKIREILIKAKKGGKSKL

SEQ ID NO: 22 - DNA, 1654 bp, Discosoma sp.
  source 1..1654: mol_type = genomic DNA
atggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaat

SEQ ID NO: 23 - protein, 239 aa, Aequorea victoria
MVSKGEELFTGVVPILVELDGDVNGHKFSVSGEGEGDATYGKLTLKFICTTGKLPVPWPTLVTTFGYGLQCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSYQSALSKDPNEKRDHMVLLEFVTAAGITLGMDELYK

SEQ ID NO: 24 - protein, 21 aa, Discosoma sp.
GSGEGRGSLLTCGDVEENPGP

SEQ ID NO: 25 - protein, 18 aa, Discosoma sp.
EGRGSLLTCGDVEENPGP

SEQ ID NO: 26 - protein, 22 aa, Discosoma sp.
GSGATNFSLLKQAGDVEENPGP

SEQ ID NO: 27 - protein, 19 aa, Discosoma sp.
ATNFSLLKQAGDVEENPGP

SEQ ID NO: 28 - protein, 23 aa, Equine rhinitis A virus
GSGQCTNYALLKLAGDVESNPGP

SEQ ID NO: 29 - protein, 20 aa, Equine rhinitis A virus
QCTNYALLKLAGDVESNPGP

SEQ ID NO: 30 - protein, 25 aa, Foot-and-mouth disease virus
GSGVKQTLNFDLLKLAGDVESNPGP

SEQ ID NO: 31 - protein, 22 aa, Foot-and-mouth disease virus
VKQTLNFDLLKLAGDVESNPGP

SEQ ID NO: 32 - protein, 46 aa, synthetic construct
  REGION 1..46: note = Synthetic construct
MAGGLNDIFEAQKIEWHEGGENLYFQGGDYKDDDDKGGAAASKVRS

SEQ ID NO: 33 - protein, 12 aa, synthetic construct
  REGION 1..12: note = Synthetic construct
MYPYDVPDYAGG

SEQ ID NO: 34 - protein, 12 aa, synthetic construct
  REGION 1..12: note = Synthetic construct
GGMYPYDVPDYA

SEQ ID NO: 35 - protein, 10 aa, synthetic construct
  REGION 1..10: note = Synthetic construct
GGWSHPQFEK

(19) United States
(12) Patent Application Publication
(10) Pub. No.: US 2026/0275329 A1
(43) Pub. Date: Sep. 17, 2026

(54) VECTORS EXPRESSING COLOR AND SELECTABLE MARKERS

(71) Applicants: THE TRUSTEES OF INDIANA UNIVERSITY, Bloomington, IN (US); THE UNITED STATES GOVERNMENT AS REPRESENTED BY THE DEPARTMENT OF VETERANS AFFAIRS, Washington, DC (US)

(72) Inventors: Uptal DAVE, Nashville, TN (US); Justin LAYER, Indianapolis, IN (US)

(21) Appl. No.: 19/691,469

(22) Filed: May 28, 2026

Related U.S. Application Data
(63) Continuation of application No. 17/276,213, filed on Sep. 18, 2019.
(60) Provisional application No. 62/733,240, filed on Sep. 19, 2018.

(51) Int. Cl. C12N 15/10; C12N 15/65; C12N 15/86

(52) U.S. Cl. CPC C12N 15/1058; C12N 15/1065; C12N 15/65; C12N 15/86; C12N 2740/15043

(57) ABSTRACT

A system for multiplex expression of proteins in eukaryotic cells is provided wherein cells simultaneously expressing each of the proteins of the system in a cell can be selected and/or visualized. The system comprises a plurality of lentiviral based vectors that each allow for the expression of the protein of interest, and a fluorescent protein linked to a fusion peptide comprising a proteolytic cleavage site that itself is linked to an antibiotic resistance protein. Each of the vectors of the system has a separate and unique fluorescent protein encoding gene and/or a separate and unique antibiotic resistance gene.

### CROSS REFERENCE TO RELATED APPLICATIONS

This application is a continuation of U.S. patent application Ser. No. 17/276,213, filed on Mar. 15, 2021, which is a U.S. national counterpart application of PCT Application No. PCT/US2019/051682 filed Sep. 18, 2019, which claims priority to U.S. Provisional Patent Application No. 62/733,240 filed on Sep. 19, 2018, the disclosure of each of which is hereby expressly incorporated by reference in their entirety.

### STATEMENT OF GOVERNMENT SUPPORT

This invention was made with government support under CA207530 awarded by the National Institutes of Health. The government has certain rights in the invention.

### INCORPORATION BY REFERENCE OF MATERIAL SUBMITTED ELECTRONICALLY

The Sequence Listing associated with this application is provided in .xml format in lieu of paper copy, and is hereby incorporated by reference into the specification. The name of the .xml file containing the sequence listing is “29920-443496SequenceListing.xml”. The .xml file is 46,152 bytes, and was created on May 28, 2026, and is being submitted electronically, concurrent with the filing of this application.

### BACKGROUND

It has become clear over the past several decades that nearly every critical function within mammalian cells is performed by molecular machines. Examples abound of such critical functions as transcription and translation, which are carried out by gigantic macromolecular complexes comprised of multiple smaller proteins. The smaller proteins, subunits of the macromolecular complexes, bind with specificity and stoichiometry to their partners and assemble into functional machines. Besides normal physiologic function, there are also prominent examples of cancer-causing proteins that do not function on their own but rather as part of complexes.

Standard molecular biology techniques allow researchers to create mutant proteins (or synthetic proteins) that can be functionally tested. However, if most of these functions require assembly into larger complexes, it may be necessary to express partners simultaneously in the same cell to fully appreciate their structure/function relationship. This is a major challenge in experimental biology.

As disclosed herein, one approach to solving this problem is to construct a multiplexed retroviral (e.g., lentiviral) expression system. Lentiviruses such as HIV-1 provide a backbone for the creation of vectors that allow transduction into an immense variety of human cells, including lines established from cancers and even primary cells freshly derived from patients. In accordance with one embodiment, a published lentiviral vector was modified to include fluorescent proteins of various colors to create a multiplexed lentiviral expression system. This multiplexed lentiviral expression system is a significant novel advance in that it combines fluorescence markers and antibiotic resistance in a high titer lentiviral backbone. We envision researchers using this vector to express multiple proteins in the same cell, or wherever it may be advantageous to have both fluorescence and antibiotic resistance to confirm and maintain expression of any cDNA cloned within the vector.

### SUMMARY

The present disclosure is directed to viral expression vectors and methods of using such vectors in a system for multiplex expression of proteins in eukaryotic cells. In one embodiment the system utilizes a retroviral backbone including for example a lentiviral based backbone. Advantageously, the system allows for the selection of eukaryotic cells that comprise multiple gene constructs and simultaneously express multiple uniquely tagged gene products, thus allowing for the study of protein interactions in vivo. In accordance with one embodiment, the system utilizes a modular retroviral shuttle vector that simultaneously expresses an exogenous gene product, a fluorescent protein, and an antibiotic resistance gene product, all from a single expression cassette. The modular design of the novel expression cassette allows for the creation of a set of vectors wherein each vector comprises a unique combination of fluorescent markers and antibiotic resistances.

In one embodiment, each retroviral vector of the system comprises its own spectrally distinct fluorescent marker gene and unique antibiotic resistance gene. Careful selection of combinatorial fluorescent markers and antibiotic markers allows for creation of stably transduced cell lines that simultaneously express multiple (3, 4, 5 or more) unique recombinant exogenously introduced gene products. Advantageously, the system allows confirmation that each of the introduced exogenous genes is retained and simultaneously expressed.

In accordance with one embodiment the modular retroviral expression vectors of the present disclosure comprise regulatory elements for gene expression, a multiple cloning site (MCS), a visible marker gene; and a selectable marker gene wherein each of said genes are operably linked to regulatory elements and are expressed from a single expression cassette. The MCS provides a convenient site for the insertion of a nucleic acid sequence of interest, including for example a sequence encoding for a protein, into the vector to operably link the inserted nucleic acid sequence to the retroviral expression elements. Upon transduction of the viral vector into a eukaryotic cell, the inserted nucleic acid sequence, detectable visible marker and the selectable marker are all expressed simultaneously. By transducing cells with multiple classes of retroviral vectors of the present disclosure (wherein the classes differ based on the nucleic acid sequence inserted into the vector and by either the visible marker or the selectable marker, or both), the expression of multiple gene products can be tracked in a cell. In one embodiment the expression vector further comprises a nucleic acid sequence encoding a proteolytic cleavage site that is linked to the visible marker gene and the selectable marker gene, wherein the visible marker gene and the selectable marker gene are expressed as a fusion peptide.

The expression vector can be prepared using viral vectors previously known to be effective delivery vehicles for transducing eukaryotic cells. This includes Adenovirus, and Adeno-associated virus (AAV) based vectors as well as any of the retroviral based vectors known to those skilled in that art. In one embodiment the expression vector comprises a retroviral backbone. Suitable retroviral vectors are known to those skilled in the art, including but not limited to, vectors derived from a gamma-retrovirus or lentivirus. In one embodiment the retroviral backbone is derived from a lentivirus.

In one embodiment the multiplex expression system of the invention is based on a lentivirus backbone to allow for optimal transduction and expression in eukaryotic cells. In accordance with one embodiment the vectors disclosed herein can be used to transduce primary cells; and thus the system can be used to express multiple proteins in primary cells. In one embodiment the visible marker is a fluorescent protein and the selectable marker is a gene encoding antibiotic resistance gene product.

In one embodiment the visible marker gene is a fluorescent protein that is expressed under the regulatory control of the 5′LTR elements of the vector and is immediately preceded by a standard internal ribosomal entry site sequence (IRES). As shown in FIG. 1, a nucleic acid of interest may be inserted into the multiple cloning site (polylinker) immediately 5′ of the IRES. In these circumstances, expression of the cloned nucleic acid of interest may be ensured by detecting the expression of the fluorescent marker. In accordance with the present disclosure, appended to the 3′ end of the fluorescent marker gene is a nucleic acid sequence encoding a proteolytic target sequence (e.g., a P2A site) followed by an antibiotic resistance gene. Thus, the fluorescent protein and the antibiotic resistance protein are initially expressed as a fusion protein, wherein the two proteins of the fusion protein are subsequently separated by proteolytic cleavage at the P2A site. This allow researchers to not only sort positive cells based on fluorescence but also to apply antibiotic selection to enrich for cells that express their cDNA of choice.

In accordance with one embodiment a kit is provided comprising a plurality of lentiviral based vectors, each isolated in a separate container, wherein each separated vector comprises its own unique visible marker and/or selectable marker relative to the other vectors of the kit. The kit can comprise 2, 3, 4, 5, 6, 7, 8, 9, 10 or more distinct lentiviral based expression vectors. Each vector comprises a restriction site or polylinker that allows a gene encoding a protein of interest to be inserted into the vector so it is operably linked to the regulatory elements necessary for transcription and translation of the gene product. Furthermore, each vector of the kit is provided with its own unique visual and/or selectable marker gene to confirm the presence of that specific construct in the cell. In accordance with one embodiment the visual marker gene is a gene encoding a fluorescent protein and the selectable marker gene is an antibiotic resistance gene. In one embodiment each vector of the kit is provided with its own unique fluorescent protein gene and its own unique antibiotic resistance gene. In one embodiment the fluorescent protein and the antibiotic resistance protein are expressed as a fusion protein that is subsequently proteolytically cleaved to separate the expressed fluorescent protein and the antibiotic resistance proteins. Peptide sequences that are susceptible to proteolytically cleavage are known to those skilled in the art (e.g., a P2A site). In one embodiment, the vector comprises a fluorescent marker gene wherein the 3′ end of the fluorescent marker gene is joined to a nucleic acid molecule encoding a P2A site which in turn is joined to the 5′ end of a nucleic acid molecule encoding an antibiotic resistance protein. Thus, the fluorescent protein and the antibiotic resistance protein are initially expressed as a fusion protein that is subsequently cleaved at the encoded P2A site to release the fluorescent protein and the antibiotic resistance protein as two separate functional proteins (see FIG. 2).

### BRIEF DESCRIPTION OF THE DRAWINGS

FIG. 1 is a schematic drawing showing DNA fragments cloned into a retroviral vector such as an H163 lentiviral vector (Unutmaz et al (1999) Journal of Experimental Medicine, 189:1735-1746). The modular aspect of the construct allows for 60 unique combinations of color and antibiotic resistance, just with the visible and selectable markers shown in the figure. Specifically there are 5 combinations each of Luciferase and antibiotic resistance and 5 combinations each of Cas9 and Antibiotic Resistance. The nucleic acid molecules used as the backbone for the vector constructs can comprise components from any of the known viruses/retroviruses arranged as necessary to produce a functional vector based on standard principles known to those skilled in the art. The cross-hatched boxes of FIG. 1 denote the long terminal repeats (LTRs) that are responsible for viral transcription. The underlined boxes (i.e. gag, pol, etc.) denote viral genes, some of which have been inactivated by deletions. Multiple vectors were constructed using the various combinations of fluorescent protein encoding genes (XFP) and the antibiotic resistance genes (AB^{R}) shown. The vectors include a multiple cloning site (MCS), providing a series of restriction endonuclease sites to provide areas for enzymatic digestion followed by ligation of a nucleic acid of interest (i.e. cDNA of choice). The vector further comprises an internal ribosomal entry site (IRES) and a sequence encoding a proteolytic cleavage site (e.g., a P2A sequence) linking the 3′ terminus of the visible marker gene (XFP) to the 5′ terminus of the antibiotic resistance gene (AB^{R}), or vice versa. Examples of fluorescent proteins and antibiotic resistance genes suitable for use in the disclosed multiplex expression vector system is provided, however the list is not exhaustive and additional compounds are known those skilled in the art and can be used in accordance with the present disclosure. Abbreviation of the antibiotic resistance genes are as follows: puromycin (PURO^{R}). Hygromycin (HYGRO^{R}), geneticin (G418^{R}), Zeocin (ZEO^{R}), and Blasticidin (BLAST^{R}).

FIG. 2 is a schematic showing DNA fragments cloned into the H163 lentiviral expression vector for expressing a HALO N-terminal tagged LMO2 protein linked to an Enhanced Green Fluorescent Protein (EGFPII) and an antibiotic resistance gene for hygromycin, wherein the encoded EGFPII and HYGRO^{R} proteins are expressed as a fusion protein linked via the self-cleaving proteolytic site P2A.

### DETAILED DESCRIPTION

### Definitions

The term “about” as used herein means greater or lesser than the value or range of values stated by 10 percent, but is not intended to designate any value or range of values to only this broader definition. Each value or range of values preceded by the term “about” is also intended to encompass the embodiment of the stated absolute value or range of values.

As used herein a “polylinker” and “Multiple Cloning Site” are used interchangeably and define a region of nucleic acid that comprises two or more unique restriction endonuclease sites.

As used herein any reference to a “2A family” sequence, or “2A family cleavage site”, absent any further designation is intended to be a generic reference to the entire 2A peptide family including but not limited to P2A (SEQ ID NO: 26 or SEQ ID NO: 27), E2A (SEQ ID NO: 28 and SEQ ID NO: 29), F2A (SEQ ID NO: 30 and SEQ ID NO: 31) and T2A (SEQ ID NO: 24 or SEQ ID NO: 25). The sequence “GSG” (Gly-Ser-Gly) on the N-terminal of a 2A peptide is optional for function. F2A is derived from foot-and-mouth disease virus 18; E2A is derived from equine rhinitis A virus; P2A is derived from porcine teschovirus-1 2A; and T2A is derived from thosea asigna virus 2A.

As used herein, the term “retroviruses” refers to viruses having an RNA genome that is reverse transcribed by retroviral reverse transcriptase to a cDNA copy that is integrated into the host cell genome. Retroviral vectors and methods of making retroviral vectors are known in the art. Briefly, to construct a retroviral vector, native viral sequences (typically the gag and pol genes) are removed or modified to produce a virus that is replication-defective. The full DNA encoding retroviral vectors is maintained in bacterial plasmids allowing their rapid expansion and purification. In order to produce virions, a viral plasmid is introduced into a packaging cell line containing the env genes (and possibly additional viral genes) but without the LTR and packaging components (Mann et al., Cell, Vol. 33:153-159, 1983). When a recombinant plasmid containing the retroviral vector, is introduced into this cell line, the viral vector is transcribed into RNA and the packaging signal sequence allows this RNA transcript of the recombinant plasmid to be packaged into viral particles, which are then secreted into the culture media. The media containing the recombinant retroviruses is then collected, optionally concentrated, and used for gene transfer.

As used herein the term “retroviral backbone” is intended to encompass the minimal regulatory elements required for transduction of a eukaryotic host cell and expression of any associated open reading frames. Transduction is the process by which foreign DNA is introduced into a cell by a virus or viral vector.

As used herein, the term “lentivirus” refers to a genus of retroviruses that are capable of infecting dividing and non-dividing cells. Several examples of lentiviruses include HIV (human immunodeficiency virus: including HIV type 1, and HIV type 2); Visna-maedi, which causes encephalitis (visna) or pneumonia (maedi) in sheep (aka MVV), the caprine arthritis-encephalitis virus (CAEV); equine infectious anemia virus (EIAV); feline immunodeficiency virus (FIV); bovine immune deficiency virus (BIV); and simian immunodeficiency virus (SIV).

As used herein, the term “vector” refers to a nucleic acid molecule capable of mediating entry of another nucleic acid molecule into a cell. Numerous vectors are known in the art including, but not limited to, linear polynucleotides, polynucleotides associated with ionic or amphiphilic compounds, plasmids, and viral vectors. Examples of viral vectors include, but are not limited to, adenoviral vectors, adeno-associated virus vectors, retroviral vectors (including lentiviral vectors), and the like.

As used herein, the term “lentiviral vector” is used to denote any form of a nucleic acid derived from a lentivirus and used to transfer genetic material into a cell via transduction. The term encompasses lentiviral vector nucleic acids, such as DNA and RNA, encapsulated forms of these nucleic acids, and viral particles in which the viral vector nucleic acids have been packaged.

As used herein the term “expression cassette” defines a nucleic acid sequence capable of expressing a particular nucleotide sequence in an appropriate host cell. The expressed nucleotides may comprise one or more protein encoding sequences that are expressed as a single transcript. The expression cassette comprises a promoter operably linked to the nucleotide sequence of interest which is operably linked to termination signals. It also typically comprises sequences required for proper translation of the nucleotide sequence.

As used herein, the term “operably linked” refers to functional linkage between a nucleic acid expression control sequence (such as a promoter, signal sequence, enhancer or array of transcription factor binding sites) and a second nucleic acid sequence. The term, “operably linked,” when used in reference to a regulatory sequence and a coding sequence, means that the regulatory sequence affects the expression of the linked coding sequence. “Regulatory sequences,” “regulatory elements”, or “control elements,” refer to nucleotide sequences that influence the timing and level/amount of transcription, RNA processing or stability, or translation of the associated coding sequence. Regulatory sequences may include promoters; translation leader sequences; introns; enhancers; stem-loop structures; repressor binding sequences; termination sequences; polyadenylation recognition sequences; etc. Particular regulatory sequences may be located upstream and/or downstream of a coding sequence operably linked thereto. Also, particular regulatory sequences operably linked to a coding sequence may be located on the associated complementary strand of a double-stranded nucleic acid molecule. When used in reference to two or more amino acid sequences, the term “operably linked” means that the first amino acid sequence is in a functional relationship with at least one of the additional amino acid sequences.

As used herein the term “visible marker gene” defines a gene that aids in the identification of a cell or organism that comprises the gene, but confers no selective advantage to the host cell/organism. Typically a visible marker gene, when expressed in a cell produces a change in optical density, color, absorption, luminescence or fluorescence that is detectable by the human eye or an optical device.

As used herein the term “selectable marker gene” defines a gene that aids in the identification of a cell or organism that comprises the gene, by conferring a selective advantage to the host cell/organism. Typically a selectable marker gene enhances the ability of a host cell to grow and multiply relative to cells lacking the selectable marker when grown in the presence of a selection agent. For example, the selectable marker gene may confer tolerance to an otherwise toxic condition or agent such as an antibiotic.

Zeocin is a formulation of phleomycin D1, a glycopeptide antibiotic and one of the phleomycins from Streptomyces verticillus belonging to the bleomycin family of antibiotics. Antibiotic genes blaT-3 to blaT-7 are variants of the structural genes for TEM-type β-lactamases (see Sougskoff et al, Reviews of Infectious Diseases, Volume 10, Issue 4, July 1988, Pages 879-884).

### EMBODIMENTS

Described herein are nucleic acid molecules for transduction, expression and monitoring of exogenous gene products in eukaryotic cells. In one embodiment a viral vector is provided for transducing eukaryotic cells and expressing a protein, wherein the presence of the vector and expression of the transgene can be monitored and selected due to the presence of a modular set of visual and selectable marker genes.

In accordance with one embodiment a multiplexed viral expression system is provided, wherein a viral backbone is used to express a modular gene cassette comprising a polylinker, a visible marker and a selectable marker. The viral expression system can be prepared using viral vectors previously known to be effective delivery vehicles for transducing eukaryotic cells. This includes Adenovirus, and Adeno-associated virus (AAV) based vectors as well as any of the retroviruses known to those skilled in that art. In one embodiment the viral expression system comprises retroviral based expression vector that comprise a retroviral backbone. Suitable retroviral vectors are known to those skilled in the art, including but not limited to vectors derived from a gamma-retrovirus or lentivirus. In one embodiment the retroviral backbone is derived from a lentivirus.

Lentiviruses such as HIV-1 provide a backbone for the creation of vectors that allow transduction into an immense variety of human cells, including lines established from cancers and even primary cells freshly derived from patients. In accordance with one embodiment a standard lentiviral vector is modified to include fluorescent proteins of various colors. These fluorescent proteins are expressed off of the 5′LTR of the vector and are preceded by a standard internal ribosomal entry site (IRES) sequence. Consistent with the present disclosure a sequence comprising an open reading frame is inserted immediately 5′ of this IRES into the MCS. Thus, expression of the cloned cDNA may be ensured by the expression of the fluorescent marker. In a further embodiment, appended to the 3′ end of the fluorescent marker, is nucleic acid encoding a P2A site that is linked to the 5′ end of an antibiotic resistance cDNA. Thus, in this embodiment the fluorescent protein and the antibiotic resistance proteins are expressed together as a fusion protein that is proteolytically separated by the P2A. This allows researchers to not only sort positive cells based on fluorescence but also to apply antibiotic selection to enrich for cells that express the cDNA inserted into the vector.

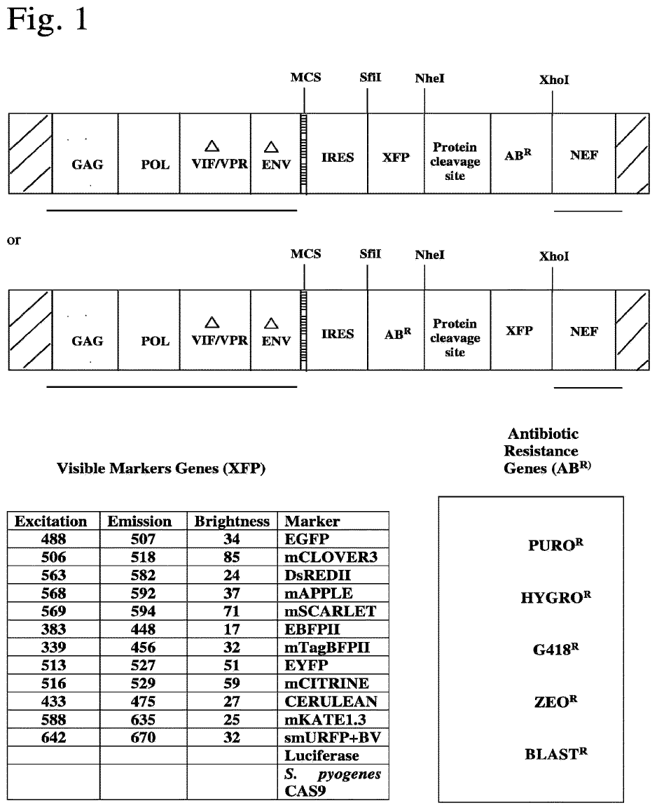

The multiplexed lentiviral expression system disclosed herein provides a significant novel advance in that it combines fluorescence markers and antibiotic resistance in a high titer retroviral backbone. In accordance with the present description a retroviral based expression vector is provided wherein the vector comprises,
a) retroviral backbone, including regulatory elements for expressing a gene;
b) a polylinker providing a convenient site for the insertion of a nucleic acid sequence of interest;
d) a visible marker gene;
e) a nucleic acid sequence encoding a proteolytic cleavage site; and
f) a selectable marker gene, wherein, the viral regulatory elements are operably linked to the polylinker, the visible marker gene and the selectable marker gene (allowing for the expression of those genes), and the nucleic acid sequence encoding a proteolytic cleavage site links the 3′ end of the visible marker gene to the 5′ end of the selectable marker gene. In one embodiment a standard internal ribosomal entry site (IRES) sequence is located 3′ to the polylinker site and 5′ to the visible marker gene. In this embodiment the vector encodes a fusion protein comprising the carboxyl terminus of the encoded visible marker protein linked via the proteolytic cleavage site to the amino terminus of the selectable marker protein. Those skilled in the art appreciate that the order of visible marker gene and selectable marker gene can be switched relative to the remaining vector elements as shown in FIG. 1. In one embodiment the visible marker gene encodes a fluorescent protein and the selectable marker gene encodes an antibiotic resistance gene. More particularly, in one embodiment the visible marker gene encodes a fluorescent protein selected from the group consisting of mCLOVER3, DsREDII, mAPPLE, mSCARLET, EBFPII, m TagBFPII, EYFP, mCITRINE, CERULEAN, mKATE1.3, SMurfBV+, firefly Luciferase, green fluorescent protein (GFP) and enhanced green fluorescent protein (EGFP), and the selectable marker gene is an antibiotic resistance gene selected from the group consisting of puromycin (PURO^{R}). Hygromycin (HYGRO^{R}), geneticin (G418^{R}), Zeocin (ZEO^{R}), and Blasticidin (BLAST^{R}).

In accordance with one embodiment the visible marker gene encodes a protein selected from the group consisting of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21 and SEQ ID NO: 23. In one embodiment, the visible marker gene encodes a protein selected from the group consisting of SEQ ID NO: 1, SEQ ID NO: 3, SEQ ID NO: 11, SEQ ID NO: 15, SEQ ID NO: 17, SEQ ID NO: 21 and SEQ ID NO: 23.

In one embodiment the retroviral vector comprises visible marker gene comprising a sequence selected from the group consisting of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22 and derivatives thereof that have been modified to comprise human codon preferences for the encoded amino acids.

Advantageously, the retroviral expression vector can comprise any combination of visible marker gene and selectable marker gene from those known to the skilled practitioner for use in eukaryotic cells. As shown in the embodiment of FIG. 1, upper construct, cleavage of the vector construct with SfiI and NheI will allow excision and replacement of the visible marker gene with any of the visible marker genes shown in FIG. 1. Furthermore, cleavage of the vector construct with NheI and XhoI will allow excision and replacement of the proteolytic cleavage sequence and selectable marker gene with a nucleic acid molecule comprising a proteolytic cleavage sequence (including any nucleic acid encoding a 2A family cleavage site) and any of the selectable marker genes shown in FIG. 1. Accordingly, a library of retroviral vectors can be generated where the presence of each vector in a transduced cell can be separately identified and selected, and each vector library member can be inserted with a unique nucleic acid of interest.

The proteolytic cleavage sequence of the retroviral vectors of the present disclosure can be selected from any known nucleic acid sequence that encodes a peptide that can be selectively cleaved after synthesis of the visible marker/proteolytic cleavage sequence/antibiotic gene fusion protein. In accordance with one embodiment the encoded proteolytic cleavage site comprises a 2A family cleavage site. More particularly, the retroviral expression vector may comprise a nucleic acid molecule that encodes a 2A family cleavage site selected from the group consisting of T2A, P2A, E2A and F2A. In one embodiment the nucleic acid molecule encoding the proteolytic cleavage site encodes a peptide sequence selected from the group consisting of SEQ ID NO: 24, SEQ ID NO: 25, SEQ ID NO: 26, SEQ ID NO: 27, SEQ ID NO: 28, SEQ ID NO: 29, SEQ ID NO: 30 and SEQ ID NO: 31. In one embodiment, nucleic acid sequence encoding the peptide cleavage site is a nucleic acid encoding a Picornavirus 2A (P2A) peptide selected from the group of SEQ ID NO: 26 and SEQ ID NO: 27.

In one embodiment the proteolytic peptide is expressed as a fusion peptide linked to a selectable marker protein. Accordingly, in one embodiment the retroviral vector comprises a nucleic acid sequence that encodes a 2A family cleavage site-antibiotic resistance fusion gene. In one embodiment the 2A family cleavage site-antibiotic resistance fusion gene is a Picornavirus 2A (P2A)-antibiotic gene, optionally wherein the antibiotic gene is selected from the group consisting of selected from the group consisting of puromycin (PURO^{R}). Hygromycin (HYGRO^{R}), geneticin (G418^{R}), Zeocin (ZEO^{R}), and Blasticidin (BLAST^{R}) genes. In one embodiment the retroviral vector comprises a Picornavirus 2A (P2A)-PURO^{R} fusion gene, Picornavirus 2A (P2A)-HYGRO^{R} fusion gene, Picornavirus 2A (P2A)-ZEO^{R} fusion gene, (P2A)-G418^{R} fusion gene or (P2A)-BLAST^{R} fusion gene.

In one embodiment the visible marker gene encodes a fluorescent protein selected from the group consisting of mCLOVER3, DsREDII, mAPPLE, mSCARLET, EBFPII, mTagBFPII, EYFP, mCITRINE, CERULEAN, mKATE1.3, SMurfBV+, firefly Luciferase and green fluorescent protein (EGFP) and the vector further comprises a nucleic acid encoding a P2A-antibiotic resistance fusion protein, wherein the nucleic acid encoding the fusion peptide is a gene construct selected from the group consisting of P2A-HYGRO^{R}, P2A-G418^{R}, P2A-ZEO^{R}, (P2A)-BLAST^{R} and (P2A)-PURO^{R}.

In accordance with one embodiment the retroviral vector can further comprise a nucleic acid sequence encoding an epitope tag. In one embodiment the nucleic acid sequence encoding the epitope tag is located immediately 5′ to the polylinker site wherein insertion of peptide coding sequence into the polylinker functionally links the epitope tag sequence to the inserted coding sequence. In this embodiment expression of the retroviral expression cassette produces a fusion peptide comprising the epitope tag linked to the N-terminus of the encoded protein of interest.

In one embodiment a kit is provided to assist in the preparation of a library of retroviral vectors wherein a plurality of nucleic acid sequences encoding for different gene products of interest are each inserted into separate unique retroviral vectors of the present disclosure. The individual retroviral vectors of the kit differ from each other only by the specific visible marker and/or selectable marker contained in the expression vector. The resulting library is produced using the kit by inserting a nucleic acid of interest into the polylinker of the expression vector. In one embodiment the nucleic acid of interest encodes a peptide or protein. The library produced using the kit comprises a plurality of expression vector classes, wherein each class comprises a different nucleic acid of interest, inserted into the polylinker, and different visible marker and/or selectable marker relative to the other expression vector classes present in the library of expression vectors.

In accordance with one embodiment a kit for preparing retroviral based nucleic acid vectors is provided wherein the kit comprises multiple classes of retroviral expression vectors separated by class into individual vessels. In one embodiment the kit comprises 2, 3, 4, 5, 6, 7, 8, 9, 10, 15 or 20 separated classes of unique expression vectors. In one embodiment the kit comprises a plurality of classes of retroviral vectors, wherein each retroviral class is provided in a separate container, and each retroviral vector class comprises
a) retroviral backbone, including regulatory elements for gene expression;
b) a polylinker;
c) a visible marker gene; and
d) a selectable marker gene, wherein, said regulatory elements are operably linked to the polylinker, the visible marker gene and the selectable marker gene, wherein each of said retroviral vectors classes differ from each other by comprising a separately identifiable visible marker gene and/or a different selectable marker. In one embodiment the retroviral vectors further comprise a nucleic acid encoding a proteolytic cleavage site, wherein the nucleic acid sequence encoding a proteolytic cleavage site links the 3′ end of the visible marker gene to the 5′ end of the selectable marker gene. In one embodiment the visible marker genes of each class of retroviral vectors encodes a fluorescent protein and the selectable marker genes of each class of retroviral vectors encodes an antibiotic resistance gene. In one embodiment each class of retroviral vectors further comprises an IRES site between the polylinker site and the selectable marker/visible marker gene. Such a vector will produce a separate protein encoded by the inserted nucleic acid of interest and a fusion peptide comprising the visible marker protein and the selectable marker protein.

In one embodiment each of the retroviral vectors of the kit further comprises a nucleic acid sequence encoding a proteolytic cleavage site that is linked to the 3′ terminus of the visible marker gene and to the 5′ terminus of the selectable marker gene and expressed as a fusion peptide. Those skilled in the art appreciate the order of the selectable marker gene and the visible marker gene in the vector is not important and the encoded fusion protein can be selectable marker/cleavage peptide/visible marker or visible marker/cleavage peptide/selectable marker. The proteolytic cleavage sequence of the retroviral vectors of the disclosed kit can be selected from any known nucleic acid sequence that encodes a peptide that can be selectively cleaved after synthesis of the visible marker/proteolytic cleavage sequence/antibiotic resistance fusion protein or selectable marker/proteolytic cleavage sequence/visible marker fusion protein. In accordance with one embodiment the encoded proteolytic cleavage site comprises a 2A family cleavage site. More particularly, the retroviral expression vector may comprise a nucleic acid molecule that encodes a 2A family cleavage site selected from the group consisting of T2A, P2A, E2A and F2A. In one embodiment the nucleic acid molecule encoding the proteolytic cleavage site encodes a peptide sequence selected from the group consisting of SEQ ID NO: 24, SEQ ID NO: 25, SEQ ID NO: 26, SEQ ID NO: 27, SEQ ID NO: 28, SEQ ID NO: 29, SEQ ID NO: 30 and SEQ ID NO: 31. In one embodiment, nucleic acid sequence encoding the peptide cleavage site is a nucleic acid encoding a Picornavirus 2A (P2A) peptide selected from the group of SEQ ID NO: 26 and SEQ ID NO: 27.

In one embodiment the proteolytic peptide is expressed as a fusion peptide linked to a selectable marker protein. Accordingly, in one embodiment the retroviral vector comprises a nucleic acid sequence that encodes a 2A family cleavage site-antibiotic resistance fusion gene. In one embodiment the 2A family cleavage site-antibiotic resistance fusion gene is a Picornavirus 2A (P2A)-antibiotic gene, optionally wherein the antibiotic gene is selected from the group consisting of selected from the group consisting of puromycin (PURO^{R}). Hygromycin (HYGRO^{R}), geneticin (G418^{R}), Zeocin (ZEO^{R}), and Blasticidin (BLAST^{R}) genes. In one embodiment the retroviral vector comprises a Picornavirus 2A (P2A)-PURO^{R} fusion gene, Picornavirus 2A (P2A)-HYGRO^{R} fusion gene, Picornavirus 2A (P2A)-ZEO^{R} fusion gene, (P2A)-G418^{R} fusion gene or (P2A)-BLAST^{R} fusion gene that can be interchangeably inserted into the expression vector of the present invention (e.g., see FIG. 1, upper construct).

In one embodiment the retroviral vectors of the kit further comprise a standard internal ribosomal entry site (IRES) sequence located 3′ to the polylinker site and 5′ to the visible marker/selectable marker genes. In one embodiment the visible marker gene encodes a fluorescent protein and the selectable marker gene encodes an antibiotic resistance protein. More particularly, in one embodiment the visible marker gene encodes a fluorescent protein selected from the group consisting of mCLOVER3, DsREDII, mAPPLE, mSCARLET, EBFPII, mTagBFPII, EYFP, mCITRINE, CERULEAN, mKATE1.3, SMurfBV+, firefly Luciferase, green fluorescent protein (GFP) and enhanced green fluorescent protein (EGFP), and the selectable marker gene is an antibiotic resistance gene selected from the group consisting of puromycin (PURO^{R}). Hygromycin (HYGRO^{R}), geneticin (G418^{R}), Zeocin (ZEO^{R}), and Blasticidin (BLAST^{R}).

In accordance with one embodiment the visible marker gene encodes a protein selected from the group consisting of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21 and SEQ ID NO: 23. In one embodiment, the visible marker gene encodes a protein selected from the group consisting of SEQ ID NO: 1, SEQ ID NO: 3, SEQ ID NO: 11, SEQ ID NO: 15, SEQ ID NO: 17, SEQ ID NO: 21 and SEQ ID NO: 23.

In one embodiment the retroviral vectors of the kit comprise visible marker genes selected from the group consisting of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22 and derivatives thereof that have been modified to comprise human cell codon preferences for the encoded amino acids.

In accordance with one embodiment a system for multiplex expression of proteins in eukaryotic cells is provided. The system comprises two or more retroviral based nucleic acid vectors wherein each retroviral vector comprises a separate and uniquely distinguishable marker for detecting the presence of the vector in the cell. In one embodiment the vectors are designed for simultaneous detection of fluorescence and antibiotic resistance, wherein each vector of the system encodes a fluorescence and antibiotic resistance gene product that can be distinguished from the other fluorescence and antibiotic resistance gene products encoded by the other vectors of the system. The number of vectors of the system is directly related to the number of proteins desired for multiplex expression. Confirmation of the expression of each multiplex protein can be confirmed by screening for a visible marker (e.g., fluorescence) and/or a selectable marker (e.g., antibiotic resistance).

According to the present disclosure, a modular system is provided that allows for multiplex expression of proteins in eukaryotic cells. The system comprises a series of retroviral expression vectors, wherein the retrovirus vectors of the system differ from each other based on the visible marker and/or selectable marker gene expressed by the individual retroviral expression vector. The system comprises at least two series/classes of retroviral vectors, and more typically 3-10 or 3-5 expression vectors. However, using various combinations of visible marker and selectable marker genes known to those skilled in the art for use in eukaryotic cells (including those disclosed in FIG. 1), a large number of unique vector constructions can be prepared. Accordingly, in one embodiment the system comprises 20, 30, 40, 50, 60, 70, 80, 90, 100 or more than 100 unique expression vectors.

In accordance with one embodiment each retroviral vector class of the system comprises
a) retroviral backbone;
b) regulatory elements for gene expression;
c) a polylinker for the insertion of a nucleic acid (e.g., cDNA) of a researcher's interest and its coding sequence;
d) a visible marker gene;
e) a nucleic acid sequence encoding a proteolytic cleavage site; and
f) a selectable marker gene,
wherein, said regulatory elements are operably linked to said polylinker, said a visible marker gene and said selectable marker gene wherein the nucleic acid sequence encoding a proteolytic cleavage site links the visible marker gene to the selectable marker gene, further wherein each of said retroviral vectors classes differ from each other by comprising a separately identifiable visible marker gene. In one embodiment the nucleic acid sequence encoding a proteolytic cleavage site links the 3′ end of the visible marker gene to the 5′ end of the selectable marker gene, and in an alternative embodiment the nucleic acid sequence encoding a proteolytic cleavage site links the 3′ end of the selectable marker gene to the 5′ end of the visible marker gene.

In accordance with one embodiment each retroviral vector class of the system comprises
a) retroviral backbone;
b) regulatory elements for gene expression;
c) a polylinker for the insertion of a coding sequence;
d) a visible marker gene;
e) a nucleic acid sequence encoding a proteolytic cleavage site; and
f) a selectable marker gene,
wherein, said regulatory elements are operably linked to said polylinker, said a visible marker gene and said selectable marker gene wherein the nucleic acid sequence encoding a proteolytic cleavage site links the visible marker gene to the selectable marker gene, further wherein each of said retroviral vectors classes differ from each other by comprising a different selectable marker gene separate and distinct from those of the other retroviral vector classes of the system. In one embodiment each of the retroviral vector classes differ from one another by having a different selectable marker gene and a different visible marker gene. In one embodiment the nucleic acid sequence encoding a proteolytic cleavage site links the 3′ end of the visible marker gene to the 5′ end of the selectable marker gene, and in an alternative embodiment the nucleic acid sequence encoding a proteolytic cleavage site links the 3′ end of the selectable marker gene to the 5′ end of the visible marker gene.

In one embodiment the retroviral backbone of the each class of retroviral vectors of the system of claim 1 or 2 wherein the is derived from a lentivirus, optionally wherein the visible marker gene is a fluorescent protein encoded by a gene selected from the group consisting of mCLOVER3, DsREDII, mAPPLE, mSCARLET, EBFPII, mTagBFPII, EYFP, mCITRINE, CERULEAN, mKATE1.3, SMurfBV+, firefly Luciferase and green fluorescent protein (EGFP); and optionally wherein the selectable marker gene is an antibiotic resistance gene selected from the group consisting of puromycin (PURO^{R}), Hygromycin (HYGRO^{R}), geneticin (G418^{R}), Zeocin (ZEO^{R}), and Blasticidin (BLAST^{R}) genes; and the nucleic acid sequence encoding a proteolytic cleavage site encodes a peptide selected from the group consisting of SEQ ID NO: 24-31.

In one embodiment, the system of the present disclosure comprises a plurality of lentiviral based vectors that each allow for the expression of the protein of interest, and a fluorescent protein linked to a fusion peptide wherein the fusion peptide comprises a proteolytic cleavage site linked to an antibiotic resistance protein. Each of the vectors of the system has a separate and unique fluorescent protein encoding gene and/or a separate and unique antibiotic resistance gene.
a) regulatory elements for gene expression;
b) a polylinker for the insertion of a coding sequence;
c) a visible marker gene; and
d) a selectable marker gene,
wherein, said regulatory elements are operably linked to said polylinker in such a manner that any coding sequence inserted into said polylinker will be operably linked to the regulatory elements, and each of the vectors of the system being provided with its own separate and distinguishable visual and/or selectable marker gene. In one embodiment the vector further comprises a nucleic acid sequence encoding a proteolytic cleavage site inserted between the visible marker gene and the selectable marker gene, wherein the visible marker protein and the selectable marker protein are expressed together but then proteolytically separated. In one embodiment the visible marker protein is a fluorescent protein and the selectable marker protein is antibiotic resistance protein.

In one embodiment the vectors of the system each comprises a different fluorescent gene selected from the group consisting of mCLOVER3, DsREDII, mAPPLE, mSCARLET, EBFPII, mTagBFPII, Enhanced yellow fluorescent protein (EYFP), mCITRINE, CERULEAN, mKATE1.3, SMurfBV+, firefly Luciferase, Green Fluorescence Protein (GFP) and Enhanced Green Fluorescence Protein (EGFP). In one embodiment the vectors of the system each comprises a different antibiotic resistance gene. In one embodiment the antibiotic resistance gene is encodes a protein that confers resistance to an antibiotic selected from the group consisting of Hygromycin B, G418, Zeocin, and Blasticidin. In a further embodiment the antibiotic resistance gene is covalently linked to a proteolytic cleavage site, including for example P2A, resulting in the production of the fusion proteins P2A-HYGRO^{R}, P2A-NEO^{R}, P2A-ZEO^{R}, P2A-BLAST^{R} and the puromycin resistance (PURO^{R}) gene.

The system of the present disclosure allows one to insert multiple nucleic acids of interests into the nuclear genome of dividing and non-dividing cells and provides a means for detecting and/or selecting for the simultaneous expression of all the inserted nucleic acids of interest. Advantageously, using the retroviral vectors of the present disclosure, the nucleic acid of interest, the visible marker and selectable marker are all transcribed under the regulatory elements of the retroviral vector backbone. There is no separate promoter for the individual genes. Therefore, the transcription of the nucleic acid of interest and the selectable and visible marker genes are linked and selecting for cells expressing the visible marker or selectable marker gene also selects for cells expressing the nucleic acids of interest.

The system comprises a series of retroviral vectors, wherein each member of the series differs from the other members of the series by the visible marker gene and/or selectable marker gene present in the vector as well as the nucleic acid of interest inserted into the vector. By inserting a different nucleic acid interest into different retroviral vectors a library of uniquely tagged expressed gene products can be transduced and inserted into the eukaryotic cell. The nucleic acid of interest comprises an open reading frame. In one embodiment the nucleic acid of interest encodes a peptide or protein.

In accordance with one embodiment a method for monitoring and maintaining the simultaneous expression of a plurality of transgenes in a eukaryotic cell is provided. In one embodiment the method comprising
inserting each of said plurality of nucleic acids of interest (e.g., transgenes) into a retroviral expression vector of the present disclosure, wherein each of said plurality of nucleic acids of interest is associated with a different visible marker gene and a different selectable marker gene relative to the other nucleic acids of interest of the plurality of transgenes to produce multiple classes of expression vectors;
introducing each of the multiple classes of expression vectors into a single cell;
selecting for cells that comprise each of the selectable markers of the multiple classes of expression vectors. In one the retroviral backbone is derived from a lentivirus; optionally the visible marker genes are fluorescent proteins encoded by a gene selected from the group consisting of mCLOVER3, DsREDII, mAPPLE, mSCARLET, EBFPII, mTagBFPII, EYFP, mCITRINE, CERULEAN, mKATE1.3, SMurfBV+, firefly Luciferase and green fluorescent protein (EGFP); optionally the selectable marker genes are antibiotic resistance genes selected from the group consisting of puromycin (PURO^{R}), Hygromycin (HYGRO^{R}), geneticin (G418^{R}), Zeocin (ZEO^{R}), and Blasticidin (BLAST^{R}); and optionally the nucleic acid sequence encoding a proteolytic cleavage site encodes a peptide selected from the group consisting of SEQ ID NO: 24-31.

As disclosed in the Examples the system of the present disclosure has be used to investigate the expression of the LMO2 oncoprotein and its protein binding partners. LMO2 is an important driver of human T-cell acute lymphoblastic leukemia (T-ALL). LMO2 does not function in isolation but functions as part of a multi-subunit complex comprised of other proteins. TAL1 and LYL1 directly bind LMO2 and also heterodimerize with class I bHLH proteins, E2A and HEB (TCF12). LMO2 also directly binds LIM domain binding protein 1 (LDB1). LDB1 in turn binds a group of proteins, Single Stranded DNA Binding Proteins 1-4. As reported in Example 2, LMO2, LDB1, SSBP3, and TAL1 or LYL1 were cloned into the multiplexed lentiviral system with each cDNA paired with a unique fluorescent marker and antibiotic genes. The resultant vectors were transduced into Jurkat T-ALL cells and the cells subjected to the relevant antibiotics to enrich for transduced cells. Each cell expressed 4 unique fluorescent colors and was resistant to antibiotics. These selected cells were lysed to verify expression of these proteins by Western blot analysis.

In accordance with embodiment 1, an expression vector is provided wherein said vector comprises:
a) retroviral backbone comprising regulatory elements;
b) a polylinker for the insertion of a nucleic acid molecule;
c) a visible marker gene;
d) a nucleic acid molecule encoding a proteolytic cleavage site; and
e) a selectable marker gene,
wherein, said regulatory elements are operably linked to said polylinker, said visible marker gene and said selectable marker gene, wherein the nucleic acid molecule encoding a proteolytic cleavage site links the visible marker gene to the selectable marker gene; optionally wherein the nucleic acid sequence encoding a proteolytic cleavage site links the 3′ end of the visible marker gene to the 5′ end of the selectable marker gene, or optionally wherein the nucleic acid sequence encoding a proteolytic cleavage site links the 3′ end of the selectable marker gene to the 5′ end of the visible marker gene.

In embodiment 2 an expression vector of embodiment 1 is provided, further comprising a single standard internal ribosomal entry site (IRES) nucleic acid sequence located 3′ to the polylinker site and 5′ to the visible marker gene and the selectable marker gene.

In embodiment 3 an expression vector of any one of embodiments 1-2 is provided wherein said visible marker gene encodes a fluorescent protein and said selectable marker gene encodes an antibiotic resistance gene.

In embodiment 4 an expression vector of any one of embodiments 1-3 is provided wherein the nucleic acid molecule encoding a proteolytic cleavage site comprises a 2A family cleavage site.

In embodiment 5 an expression vector of any one of embodiments 1~4 is provided, wherein the nucleic acid molecule encoding a proteolytic cleavage site encodes a peptide selected from the group consisting of SEQ ID NO: 24-31.

In embodiment 6 an expression vector of any one of embodiments 1-5 is provided, wherein the retroviral backbone is derived from a lentivirus.

In embodiment 7 an expression vector of any one of embodiments 1-6 is provided, wherein the visible marker gene encodes a fluorescent protein selected from the group consisting of mCLOVER3, DsREDII, mAPPLE, mSCARLET, EBFPII, mTagBFPII, EYFP, mCITRINE, CERULEAN, mKATE1.3, SMurfBV+, firefly Luciferase, green fluorescent protein (GFP) and enhanced green fluorescent protein (EGFP).

In embodiment 8 an expression vector of any one of embodiments 1-7 is provided, wherein the selectable marker gene is an antibiotic resistance gene encoding a protein conferring resistance to an antibiotic selected from the group consisting of puromycin (PURO). Hygromycin (HYGRO), geneticin (G418), Zeocin (ZEO), and Blasticidin (BLAST).

In embodiment 9 an expression vector of any one of embodiments 1-8 is provided, wherein the vector further comprises a nucleic acid molecule encoding an amino terminal epitope tag operably linked to the polylinker or the selectable or visible marker genes.

In embodiment 10 a kit for preparing multiplexed retroviral based nucleic acid vectors is provided, wherein said kit comprises a plurality of expression vector classes, wherein each retroviral vector class is contained in a separate container and comprises
a) retroviral backbone comprising regulatory elements for gene expression;
b) a polylinker;
c) a visible marker gene;
d) a nucleic acid molecule encoding a proteolytic cleavage site; and
e) a selectable marker gene,
wherein, said regulatory elements are operably linked to said polylinker, said visible marker gene and said selectable marker gene, wherein the nucleic acid molecule encoding a proteolytic cleavage site links the visible marker gene to the selectable marker gene, further wherein each of said retroviral vectors classes differ from each other by comprising a separately identifiable visible marker gene or a different selectable marker gene; optionally wherein the nucleic acid sequence encoding a proteolytic cleavage site links the 3′ end of the visible marker gene to the 5′ end of the selectable marker gene, or optionally wherein the nucleic acid sequence encoding a proteolytic cleavage site links the 3′ end of the selectable marker gene to the 5′ end of the visible marker gene.

In embodiment 11 a kit according to embodiment 10 is provided, wherein said visible marker gene encodes a fluorescent protein and said selectable marker gene encodes an antibiotic resistance gene.

In embodiment 12 a kit according to embodiment 10 or 11 is provided, wherein the nucleic acid molecule encoding a proteolytic cleavage site encodes a peptide comprising a 2A family cleavage site.

In embodiment 13 a kit according to any one of embodiments 10-12 is provided, wherein the nucleic acid molecule encoding a proteolytic cleavage site encodes a peptide selected from the group consisting of SEQ ID NO: 24-31.

In embodiment 14 a kit according to any one of embodiments 10-13 is provided, wherein the retroviral backbone is derived from a lentivirus.

In embodiment 15 a kit according to any one of embodiments 10-14 is provided, wherein the visible marker gene encodes a fluorescent protein selected from the group consisting of mCLOVER3, DsREDII, mAPPLE, mSCARLET, EBFPII, mTagBFPII, EYFP, mCITRINE, CERULEAN, mKATE1.3, SMurfBV+, firefly Luciferase, green fluorescent protein (GFP) and enhanced green fluorescent protein (EGFP).

In embodiment 16 a kit according to any one of embodiments 10-15 is provided, wherein the selectable marker gene is an antibiotic resistance gene that encodes a protein conferring resistance to an antibiotic selected from the group consisting of puromycin (PURO). Hygromycin (HYGRO), geneticin (G418), Zeocin (ZEO), and Blasticidin (BLAST).

In embodiment 17 a system for multiplex expression of proteins in eukaryotic cells is provided wherein, said system comprises a plurality of retroviral based nucleic acid vector classes wherein each retroviral vector class comprises
a) retroviral backbone;
b) regulatory elements for gene expression;
c) a polylinker for the insertion of a nucleic acid molecule;
d) a visible marker gene;
e) a nucleic acid molecule encoding a proteolytic cleavage site; and
f) a selectable marker gene,
wherein, said regulatory elements are operably linked to said polylinker, said visible marker gene and said selectable marker gene wherein the nucleic acid molecule encoding a proteolytic cleavage site links the visible marker gene to the selectable marker gene, further wherein each of said retroviral vectors classes differ from each other by comprising a separately identifiable visible marker gene or a different selectable marker gene.

In embodiment 18 a system according to embodiment 17 is provided, wherein each of said plurality of retroviral vector classes comprises a unique selectable marker gene as well as a separately identifiable visible marker gene relative to those of the other retroviral vector classes.

In embodiment 19 a system according to any one of embodiments 17-18 is provided, wherein the system comprises three or more retroviral vectors classes.

In embodiment 20 a system according to any one of embodiments 17-19 is provided, wherein the retroviral backbone is derived from a lentivirus;
the visible marker gene is a fluorescent protein encoded by a gene selected from the group consisting of mCLOVER3, DsREDII, mAPPLE, mSCARLET, EBFPII, mTagBFPII, EYFP, mCITRINE, CERULEAN, mKATE1.3, SMurfBV+, firefly Luciferase and green fluorescent protein (EGFP);
the selectable marker gene is an antibiotic resistance gene selected from the group consisting of puromycin (PUROR). Hygromycin (HYGROR), geneticin or neomycin (G418R), Zeocin (ZEOR), and Blasticidin (BLASTR) genes; and
the nucleic acid molecule encoding a proteolytic cleavage site encodes a peptide selected from the group consisting of SEQ ID NO: 24-31.

In embodiment 21 a method for monitoring and maintaining the simultaneous expression of a plurality of transgenes in a eukaryotic cell is provided, wherein said method comprises
inserting each of said plurality of transgenes into an expression vector of embodiment 1 wherein each of said plurality of transgenes is associated with a different visible marker gene and a different selectable marker gene relative to the other transgenes of the plurality of transgenes to produce multiple classes of expression vectors;
introducing each of the multiple classes of expression vectors into a single cell;
selecting for cells that comprise each of the visible markers or each of the selectable markers of the multiple classes of expression vectors.

In embodiment 22 a method in accordance with embodiment 21 is provided, wherein the retroviral backbone is derived from a lentivirus;
the visible marker genes are fluorescent proteins encoded by a gene selected from the group consisting of mCLOVER3, DsREDII, mAPPLE, mSCARLET, EBFPII, mTagBFPII, EYFP, mCITRINE, CERULEAN, mKATE1.3, SMurfBV+, firefly Luciferase and green fluorescent protein (EGFP);
the selectable marker genes are antibiotic resistance genes selected from the group consisting of puromycin (PUROR). Hygromycin (HYGROR), geneticin or neomycin (G418R), Zeocin (ZEOR), and Blasticidin (BLASTR); and
the nucleic acid molecule encoding a proteolytic cleavage site encodes a peptide selected from the group consisting of SEQ ID NO: 24-31.

### Example 1

### Construction of Lentiviral Vectors

### Method of Construction

We assembled in silico an artificial DNA fragment containing the publically available sequences for Encephalomyocarditis virus internal ribosomal entry site (IRES) sequence, enhanced green fluorescent protein (EGFP) cDNA, and puromycin resistance (Puro^{r}) gene as follows:

A 5′ EcoRI site preceded the IRES sequence, which was immediately followed by an SfiI site flanking the 5′ end of EGFP coding sequence. The initiator methionine codon (i.e. ATG) of EGFP was embedded in the SfiI site. The codon for the last amino acid of EGFP was immediately followed by an NheI site, which immediately preceded the 5′ end of an artificial cDNA encoding a human-codon optimized Picornavirus 2A (P2A)-Puro^{r} fusion gene. The Picornavirus 2A is a self-cleaving site. An XhoI site immediately followed the stop codon of the P2A-Puro^{r} cassette. This sequence was submitted to Integrated DNA Technologies (Coralville, Iowa) for synthesis using their gBlock method. The newly synthesized double standed DNA was digested with EcoRI and XhoI and ligated to EcoRI/XhoI digested pBluescript SK (+) plasmid (Stratagene). Multiple clonal isolates were subjected to automated DNA sequencing with 5′ M13R and 3′ T7 promoter primers to verify that the sequence synthesized by IDT matched what we had constructed in silico. A single clone perfectly matching our DNA sequence was digested with EcoRI and XhoI to release it from the pBluescript plasmid. This insert was isolated by gel purification and ligated to EcoRI/XhoI digested lentiviral backbone, pH110 (provided by Dr. Derya Unutmaz, Jackson Laboratories Institute for Genomic Medicine, Hartford, Connecticut). We referred to the resulting construct as pH163-EGFP-Puro^{r} which was maintained and propagated in XL1 E. coli.

Functionality of the construct was tested by transfection of pH163-EGFP-Puro^{r} into 293T cells with pVSV-G and collection of viral supernatant. This viral supernatant was then used for transduction of Jurkat cells; procedures were performed as previously described (Layer J H, Alford C E, McDonald W H, and Dave U P. LMO2 Oncoprotein Stability in T-Cell Leukemia Requires Direct LDB1 Binding. Mol Cell Biol. 2016; 36(3):488-506). Transduction efficiency was quantified based on proportion of Jurkat cells that became EGFP positive, as measured by fluorescence microscopy and flow cytometry. We also tested the functionality of the Puro^{r} expression since transduced Jurkat cells became resistant to the cytotoxic poison puromycin.

Once verified to be functional, pH163-EGFP-Puro^{r} was used to create additional vectors encoding different combinations of fluorescence markers, as shown in FIG. 1. SfiI/NheI fragments corresponding to mCLOVER3, DsREDII, mAPPLE, mSCARLET, EBFPII, mTagBFPII, EYFP, mCITRINE, CERULEAN, mKATE1.3, SMurfBV+, or firefly Luciferase were designed in silico such that non-coding substitutions were made to eliminate any internal NotI, EcoRI, SfiI, NheI, or XhoI sites. Codons were also optimized for human adaptive index on a case-by-case basis, as necessary. mSCARLET, mTagBFPII, mKATE1.3, and SMurfBV+ fragments also included DNA sequences encoding an amino terminal V5 epitope tag, which would allow detection of the recombinant protein in cellular extracts via Western blotting. DNA sequences were submitted to IDT as above for synthesis. The synthesized double stranded DNA from IDT was then digested with SfiI/NheI and used to replace the equivalent EGFP fragment from pH163-EGFP-Puro^{r}. Insert DNA was verified by automated DNA sequencing, and constructs were tested for functionality as described above, according to expression of the respective fluorescent protein, along with resistance to puromycin. The work resulted in the creation of pH163-(color)-Puro^{r}.

Next, we worked towards replacing the Puro^{r} with alternative antibiotic resistance cDNAs. We designed in silico NheI/XhoI fragments corresponding to P2A-hygro^{r}, P2A-G418^{r}, P2A-Zeo^{r}, P2A-Blat^{r}. These cDNAs correspond to proteins that confer resistance to hygromycin, G418 (or neomycin), zeocin, and blasticidin, respectively, all familiar antibiotics to those working with mammalian cells in tissue culture. As above, the synthetic DNAs were digested and used to replace the equivalent P2A-Puro^{r} cassette in pH163-EGFP-Puro^{r}. Individual clonal constructs were validated for their ability to produce functional virus, and for their ability to transduce Jurkat cells, and for their ability to express fluorescent colors and resistance to hygromycin B, G418, zeocin, or blasticidin, respectively.

Each pH163-(color) vector is available with various antibiotic resistance genes as shown, representing a total of 45 vectors that have been constructed at this writing. The vectors are plasmids with ampicillin resistance and may be maintained in E. coli in the presence of ampicillin.

### Example 2

### Use of Multiplex Lentiviral Expression Vectors to Investigate the Role of LDB1 on Oncoprotein Partners in T-Cell Leukemia

In hematopoiesis, lineage-specific transcription factors control specification of the hematopoietic stem cell (HSC) towards multiple diverse cell types. At the top of this developmental hierarchy are approximately 9 factors that directly affect the HSC itself: BMI1, RUNX1, GATA2, LMO2, TAL1, LDB1, MLL, GFI1, and ETV6. These master regulators are conserved among all vertebrates and have been experimentally characterized in mice, zebrafish, and humans. Knockouts of any one of the genes encoding these factors causes the loss of all hematopoiesis, both embryonic and adult, by perturbing the creation, survival, or self-renewal of primitive and definitive HSCs. In examining this gene list, there are three emerging themes: First, the factors are part of a transcriptional network with autoregulation and inter-regulation; second, the factors are frequently co-opted in human leukemias by various genetic mechanisms like chromosomal translocation; and, third, all the factors function as part of multi-subunit protein complexes. Four of the factors listed above act in concert within a remarkable macromolecular complex, the LMO2/LDB1/TAL1/GATA2 (or the LDB1/LMO2) protein complex. There are diverse data supporting the idea that these proteins are bound together including co-immunoprecipitation (co-IP), co-purification followed by mass spectrometry, electrophoretic mobility shift assays, and co-occupancy at target genes by chromatin immunoprecipitation.

The assembly of the LDB1/LMO2 complex depends upon specific interactions between LMO2 and class II bHLH proteins, LMO2 and GATA factors, and LMO2 and LDB1. There are multiple bHLH and GATA paralogs capable of binding LMO2 so multiple versions of the LMO2-associated complex exist depending upon the expression of the subunits. LMO2 is an 18 kDa protein with two Zinc-binding LIM domains, LIM1 and LIM2. LIM1 folds to create an interface for binding class II bHLH proteins such as TAL1 and LYL1. LIM2 has an interface that binds GATA factors 1-3. A portion of LIM1 also serves as an interface for binding to the LIM interaction domain (LID) of LDB1. LDB1 has a self-association domain through which LDB1 may dimerize or multimerize. The class II bHLH proteins heterodimerize with class I bHLH proteins such as E2.2, E12, E47, and HEB. The bHLH proteins and GATA proteins can be part of the same complex allowing the LDB1/LMO2 complex to bind adjacent E boxes and GATA sites. Such motifs bound by LMO2/LDB1 complexes have been described in erythroid progenitor cells at various gene targets including the beta globin gene promoters and the locus control region (LCR). The self-association domain of LDB1 mediates looping and proximity between the beta globin LCR and beta globin proximal promoters, a seminal example of enhancer-promoter communication.

Several iterations of the LDB1/LMO2 complexes are drivers in leukemia. In fact, LMO2 and TAL1 were originally cloned from chromosomal translocations in T-cell acute lymphoblastic leukemia (T-ALL). LMO2 was also the target of insertional activation in gammaretroviral gene therapy-induced T-ALL. Mouse modeling and the characterization of the LMO2-associated complexes have been highly informative in dissecting the pathogenesis of LMO2-induced T-ALL, underscoring the role for specific bHLH and GATA factors as requisite co-operating drivers. We recently confirmed by purification of FLAG-LDB1 and mass spectrometry that the LMO2/LDB1 complex in T-ALL closely resembles the complex hypothesized to function in normal HSCs.

Regardless of the variation in bHLH or GATA factors or the cofactors that these transcription factors may recruit, the core subunits of LMO2 and LDB1 are constant. We probed the LMO2/LDB1 interaction and discovered a discrete motif within the LDB1 LID that was essential for LMO2 binding. We consistently observed an increase in steady state abundance of LMO2 with co-expression of LDB1 and a decrease in abundance with the co-expression of LDB1ΔLID. Remarkably, this effect was observed in multiple leukemic cells including models for AML, which is consistent with recent studies showing the essentiality of LMO2 and LDB1 in these leukemias. To more closely analyze the effects on protein stability, we sought to understand the kinetics of turnover of LMO2 and its partner proteins. Towards this end, we devised a pulse chase technique through the use of multiplexed lentiviral expression of Halo-tagged proteins (Los et al., (2008) ACS chemical biology 3, 373-382.). We discovered that there is a hierarchy of protein turnover for the subunits of the complex with LDB1 being the most stable protein. Furthermore, we discovered that every subunit, including both direct and indirect binding partners of LDB1, were stabilized by LDB1. These findings have remarkable implications for the assembly of this important macromolecular complex and underscore LDB1 as the major core subunit that could be targeted in leukemias.

### Development of a Novel Multiplexed Lentiviral Expression Vector System

Previously we used multiplexed lentiviral infection with GFP- and RFP-marked viruses to create recombinant leukemia cell lines, in conjunction with fluorescence assisted cell sorting (FACS) (Layer et al., (2016) Mol Cell Biol 36, 488-506). FACS sorting was laborious and expensive, while the use of GFP and RFP markers limited the number of co-expressed recombinant factors to two (LDB1 and LMO2). Moreover, we observed that initially homogenous FACS-sorted cell lines could inactivate transgene (GFP or RFP) expression over time, consistent with either transgene silencing or competitive advantage/outgrowth of low-expressing clones. This phenomenon occurred variably amongst different cell lines/types.

To circumvent these limitations for the present study, we designed a suite of novel lentiviral vectors. This modular vector family expresses additional fluorescence protein markers that are spectrally distinct, allowing multiplexed co-infection with five or more different viruses. Each vector also encodes a unique antibiotic resistance marker to allow for positive selection of transduced cells. Antibiotic resistance of transduced cells foregoes the need for FACS, and disallows transgene silencing within transduced cell lines; all of which can be proven by antibiotic-enforced consistency of fluorescence marker expression, as monitored by flow cytometry.

### Lentiviral Vector Construction

We modified a previously described second generation lentiviral vector (Unutmaz et al., (1999) 101084/jem20050075 189, 1735-1746). First, an artificial DNA fragment containing the encephalomyocarditis virus internal ribosomal entry site (IRES) sequence, enhanced green fluorescent protein (EGFP) cDNA, and puromycin resistance (PURO) cDNA were assembled in silico using publicly available DNA sequences, as follows. A 5′ EcoRI site preceded the IRES sequence, which was immediately followed by a SfiI site flanking the 5′ end of EGFP coding sequence. The initiator methionine codon of EGFP was embedded in the SfiI site. The codon for the last amino acid of EGFP was immediately followed by an NheI site, which immediately preceded the 5′ end of an artificial cDNA encoding human-codon optimized Picornavirus 2A (P2A)-PURO resistance fusion gene. An XhoI site immediately followed the stop codon of the P2A-PURO cassette. This fragment was synthesized as a G Block by Integrated DNA Technologies (IDT, Coralville, Iowa). Synthetic DNA was digested with EcoRI and XhoI and ligated to equivalently digested pBluescript SK (+) (Stratagene). Multiple clonal isolates were subjected to automated DNA sequencing with 5′ M13R and 3′ T7 promoter primers. A single clone perfectly matching the DNA sequence was digested preparatively with EcoRI and XhoI; liberated insert was isolated and ligated to equivalently digested pH110 (Unutmaz et al., 1999). The resultant construct is referred to as pH163-EGFP-PURO.

Functionality of pH163 EGFP PURO was first tested for production of virus that could transduce Jurkat cells to EGFP positivity and puromycin resistance (see details below), and the vector backbone was subsequently used as a basis to create additional constructs encoding different combinations of fluorescence markers and antibiotic resistances, as follows. SfiI/NheI fragments corresponding to mCLOVER3, DsREDII, mAPPLE, mSCARLET, EBFPII, mTagBFPII, EYFP, mCITRINE, CERULEAN, mKATE1.3, SMurfBV+, firefly Luciferase, or S. pyogenes Cas9 were designed in silico such that non-coding substitutions were made to eliminate any internal NotI, EcoRI, SfiI, NheI, or XhoI sites. Codons were also optimized for human adaptive index on a case-by-case basis, as necessary. mCLOVER3, mSCARLET, mTagBFPII, mKATE1.3, and SMurfBV+ fragments also encoded an amino terminal V5 epitope tag, useful for detection of the recombinant protein in cellular extracts via western blotting. Synthetic G Block DNA was digested with SfiI/NheI and use to replace the equivalent EGFP fragment from H163 EGFP PURO. Insert DNA was verified by automated DNA sequencing, and constructs were tested for functionality according to viral production and transduction/expression within Jurkat cells of the respective fluorescent protein, along with resistance to puromycin.

NheI/XhoI fragments corresponding to P2A-HYGRO, P2A-NEO, P2A-ZEO, and P2A-BLAST were designed in silico according to the above considerations, and synthetic DNAs were used to replace the equivalent P2A-PURO cassette in H163-EGFP-PURO. Individual clonal constructs were validated/tested for ability to produce virus functional for transduction of Jurkat cells to EGFP positivity and resistance to Hygromycin B, G418, Zeocin, or Blasticidin, respectively.

Individual clones conferring the appropriate fluorescent protein expression in combination with PURO selection, or antibiotic resistance companion with EGFP expression, were used to isolate the functionally validated and relevant SfiI/NheI or NheI/XhoI fragment. The isolated functional DNA fragments were used to reconstitute the desired combination of fluorescent marker and antibiotic resistance in the H163 vector backbone, as depicted in FIG. 1.

cDNAs and Tagged Constructs

Subcloning of the 375 amino acid (aa) human LDB1 cDNA was described previously (Layer et al., 2016); wild type cDNA and mutant derivatives were arranged as either 5′ NotI/3′ EcoRI or 5′ BamHI/3′ EcoRI fragments. Vector-embedded epitope tags appended to LDB1 constructs were N-terminal and were either tandem biotin acceptor domain (BAD)/FLAG (MAGGLNDIFEAQKIEWHEGGENLYFQGGDYKDDDDKGGAAASKVRS; SEQ ID NO: 32) or HAx1 (MYPYDVPDYAGG; SEQ ID NO: 33). The 158 aa wild type human LMO2 cDNA or mutant derivatives were synthesized as G Blocks with tandem 5′ NotI/BamHI and 3′ EcoRI sites and ligated into NotI/EcoRI digested pBluescript II SK (+). The LMO2 cDNA encoded tandem C-terminal HA (GGMYPYDVPDYA; SEQ ID NO: 34) and SII (GGWSHPQFEK; SEQ ID NO: 35) tags. cDNAs encoding wild type or mutant human 331 aa TAL1, 280 aa LYL1, 361 aa SSBP2, and 388 aa SSBP3 were all synthesized as G Blocks with 5′ NotI/BamHI and 3′ EcoRI sites and ligated into NotI/EcoRI digested pBluescript II SK (+). Sequence encoding N-terminal HAx1 tag (MYPYDVPDYAGG; SEQ ID NO: 33) was located between the 5′ NotI and BamHI sites, and the BamHI site immediately preceded the natural initiator methionine codon. In order to create Lentiviral vectors encoding subunits with BAD/FLAG, HA/SII, or HAx1 tags, clonally-derived NotI/EcoRI fragments encoding BAD/FLAG-LDB1, LMO2-HA/SII, HAx1-TAL1, HAx1-LYL1, HAx1-SSBP2, or HAx1-SSBP3 were transferred from pBluescript II SK (+) vectors into likewise digested H163 vectors. The N-terminal 312 aa Halo tag sequence was PCR amplified from His₆HaloTag® T7 Vector pH6HTN (Promega) as a 5′ SpeI, 3′ BamHI/EcoRI fragment and ligated into SpeI/EcoRI digested pBluescript II SK (+); the resultant vector was named pHalo-tag-N. Tandem TGA stop codons were located between the BamHI and EcoRI sites. N-terminal HALO fusion constructs were created by ligating clonally-derived BamHI/EcoRI fragments encoding LDB1, LMO2, TAL1, LYL1, SSBP2, or SSBP3 into equivalently digested pHalo-tag-N.

In order to create lentiviral vectors encoding N-terminal HALO fusions, NotI/EcoRI fragments were recovered from these pHalo-tag-N vectors and ligated into likewise-digested H163 vectors in order to create H163-Halo-tag-N subunit vectors. All recombinant DNA manipulation and propagation utilized E. coli XL1 Blue. All clonal inserts were verified in their entirety by automated DNA sequencing. All mutant derivatives used optimal human codons to encode amino acid substitutions. Maxipreps of lentiviral vector DNA for transfection/virus production were prepared by a modified alkaline lysis/lithium chloride/PEG precipitation protocol in conjunction with extensive phenol/chloroform extraction and ethanol precipitation.

### Cell Lines, Tissue Culture, Recombinant Lentiviruses, Transductions, and Production of Stable Cell Lines

HEK 293T, Jurkat, K562, U937, KOPT-K1, and LOUCY cells were acquired from the American Type Culture Collection (ATCC). HEK293T cells were cultured in Iscove's modified Dulbecco's medium (IMDM)-10% fetal bovine serum (FBS), and other lines were cultured in RPMI 1640-10% FBS, at 37° C. in 5% CO₂. Log-phase HEK 293T cells in 10-cm dishes containing 10 ml medium and 5×10⁶ to 8×10⁶ cells were transfected by a calcium phosphate-HEPES-buffered saline method with 1 μmol pH163 constructs and 2 μmol pMD-2 for producing pseudotyped lentiviruses. At 12 to 18 h posttransfection, medium was aspirated and replaced with 6 ml fresh medium, which was harvested and replaced at 24 h and 48 h. Media containing viral particles was aliquoted and frozen at −80° C. and viral titer was subsequently estimated by serial dilution infection of Jurkat cells. Varying volumes of viral supernatant were mixed with 5×10⁶ to 1×10⁷ log phase Jurkat cells in a final volume of 10 ml within a T-25 flask (Eppendorf) and subsequently cultured for 72 hours, at which time percentage of fluorescence-positive cells was first roughly determined using an EVOS FL inverted fluorescence microscope (Invitrogen), and then precisely determined using a CytoFLEX benchtop cytometer (Beckman). Microscopy and Cytometry gating parameters were established using parallel culture of non-infected cells as reference. A multiplicity of infection (MOI) of 1 was associated with a fluorescence-positivity of 30% or less. Typical viral titers were 1-2×10⁶ infectious particles per milliliter. Jurkat cells infected at an MOI of 1-2 were expanded into a 50 ml culture containing antibiotics to eliminate non-infected cells. Antibiotic regimen and dose varied depending upon the selectable marker encoded by the virus in question and the cell line being transduced; antibiotic concentration kill curves were empirically established for naïve cell lines. As an example, typical antibiotic concentrations for transduced Jurkat cells were puromycin at 2 μg/ml, hygromycin B at 200 μg/ml, G418 at 500 μg/ml, Blasticidin at 10 μg/ml, or Zeocin at 50 μg/ml. After 4-10 days of antibiotic selection cell populations were typically 100% fluorescence positive, at which point they were cryo-preserved in liquid nitrogen using growth media supplemented with 10% DMSO, subjected to iterative rounds of transduction with additional viruses exactly as described above, or used directly for experiments.

### Whole-Cell Extract, Immunoprecipitations, Antibodies, and SDS-PAGE/Western Blotting

Late-log-phase cultures of ~7.5×10⁷ cells were harvested by centrifugation at 800×g for 10 min, and cell pellets were washed with PBS (phosphate-buffered saline) (2.7 mM KCl, 1.47 mM KH₂PO₄, 8.1 mM Na₂HPO₄, 137 mM NaCl) and resuspended in 500-1000 μl extraction buffer (20 mM HEPES [pH 7.6], 300 mM NaCl, 20 mM imidazole, 0.1% Triton X-100, 10% glycerol, and protease inhibitor cocktail (Thermo/Pierce)). Cells were disrupted by mild sonication with the microtip of a Branson model 250 sonifier on the low-power setting, and the soluble extract was clarified by centrifugation at 14,000×g for 15 min. Extract protein content was typically 5 to 10 μg/μl. A portion was mixed with an equal volume of 2×SDS sample buffer and briefly heated to 75° C. For immunoprecipitations (IP), 100 μl of soluble extract was supplemented with an additional 100 μl of extraction buffer also containing 5 μl anti-FLAG M2 resin (catalog number A2220; Sigma) or 5 μl of Protein A/G resin (Santa Cruz) along with 1-2 micrograms of anti-LMO2 IgG, then rocked at 4° C. for 3 to 4 h. Immune complexes were isolated by centrifugation, washed 3 times with 200 μl of extraction buffer, and eluted by heating with 100 μl SDS sample buffer. Samples were stored at −80° C. and briefly heated again at 75° C. just prior to loading onto handcast discontinuous SDS-PAGE gels with a 4% acrylamide stacking gel and a 4-to-15% linear gradient resolving gel (37.5%/1.0% [wt/vol] acrylamide-bisacrylamide), run at 15 V/cm for 90-105 min. Gels were transferred onto a 0.2-μm polyvinylidene difluoride (PVDF) membrane (catalog number 10600022; GE) at 50 V for 2.5 h; filters were blocked in PBS-2% non-fat dry milk (NFDM, Marsh FoodClub) and incubated with antibodies in blocking buffer overnight at 4° C.

The following antibodies for Western blotting were used according to the manufacturer's specifications: mouse monoclonal anti LDB1 IgG (catalog number sc-376030x; Santa Cruz) (detected with a goat anti mouse IgG Fc-horseradish peroxidase (HRP) conjugate, catalog number 31439; Thermo/Pierce), anti FLAG-HRP conjugate (catalog number A8592; Sigma), anti HA-HRP conjugate (catalog number 12013819001; Roche), anti V5-HRP conjugate (to detect mSCARLET and other V5 tagged fluorescent proteins, catalog number 46-0708, Invitrogen), rabbit polyclonal anti TAL1 IgG (catalog number A305-300A, Bethyl), (detected with a goat anti rabbit IgG-HRP conjugate [catalog number 211-032-171; Jackson ImmunoResearch]), mouse monoclonal anti SSBP2 IgG (catalog number sc-166687, Santa Cruz), mouse monoclonal anti HALO IgG (catalog number G921A, Promega), mouse monoclonal anti GFP IgG (catalog number 11814460001; Roche), rabbit polyclonal anti tubulin IgG (catalog number SC-9104; Santa Cruz). The high-affinity/sensitivity/specificity mouse anti valosin-containing protein (anti VCP) antibody (catalog number ab11433; Abcam) was used for multiplex Western blotting as a loading control. The 1A93B11 mouse anti LMO2 IgG was described previously (Layer et al., 2016). Western blots were developed with enhanced chemiluminescense (ECL) detection (SuperSignal Pico West Plus, catalog number 1863099, Thermo/Pierce). All images were obtained within the linear signal detection range using a ChemiDoc Touch imaging system (BioRad). Images were analyzed using ImageLab Software version 5.2.1 (BioRad) and exported to Adobe Photoshop and Illustrator for figure assembly.

### HaloLife Assay: Live Cell Pulse Chase Analysis

1.25 10⁵ cells were collected from log-phase cultures by centrifugation at 1,200×g for 1 min. The culture media was removed, and cells were resuspended with 125 μL RPMI containing 10% FBS and HaloTag Ligand R110 (Promega Ca.) at a final concentration of 100 nM, per the company's instructions. The resuspended cells were then incubated for 90 min at 37° C. in 5% CO₂. After 90 min the cells were centrifuged at 12,000×g for 1 min and washed with PBS (2.7 mM KCl, 1.47 mM KH₂PO₄, 8.1 mM Na₂HPO₄, 137 NaCl) containing 0.1% BSA (bovine serum albumin) a total of 3 times to remove excess HaloTag Ligand R110. Cells were resuspended in 600 μL RPMI containing 10% FBS, and 4, 150 μL aliquots were transferred to a 96-well round-bottom plate (TPP). 10,000 events were then immediately analyzed from 1 of the 4 150 μL aliquots using a CytoFLEX benchtop cytometer (Beckman). All subsequent chase time points were collected using this initial analysis as a reference. Between flow cytometry analyses, the 96-well plate containing the HaloTag Ligand R110 labeled cells were placed in an incubator at 37° C. with 5% CO₂ until the next collection point. Flow cytometry analyses were collected 3, 4, and 5 hours after TO for all cells, with the exception those containing Halo-tagged LDB1 and LYL1 due to their significantly different observed half-lives. For cells containing Halo-tagged LDB1, flow cytometry events were recorded at 6, 12, and 24 hours after TO, and analyses were recorded 1, 2, and 3 hours after the initial time point for cells containing Halo-tagged LYL1. Replicate experiments were done on consecutive days.

### Pulse-Chase FCS File Analysis

All FCS files were analyzed using Flowjo 10.3 analysis software (FLOWJO, LLC,OR). To identify cells that were co-expressing EBFPII and/or mScarlet in conjunction with Halo-tagged proteins, non-transduced unstained Jurkat cells were used to establish a gating sequence. Their physical dimensions were grouped on an FSC-A/FSC-H plot to determine the total number of lymphocytes within the event population. A gate was then established on an FSC-A/SSC-A plot to select for live cells within the total lymphocyte population. The resulting population was then gated as a negative control for both fluorescence markers on a PB450-A (EBFPII)/FITC-A (HaloTag R110) plot. This gating sequence was then applied to all FCS files within the same experiment.

### Half-Life Calculations

Log-linear regression curves were calculated from flow cytometry analysis data to calculate Halo-tagged protein half-lives. PB450-A (EBFPII) and FITC-A (HaloTag R110 Ligand) double positive events were calculated as a percentage of the parent population for all time points collected. Replicate data for each time point was averaged, and then normalized to the initial time point. The natural log was calculated for each of the averages, and the resulting values were represented over time on a 2-dimensional scatter plot. A trend line was calculated, and the resulting slope was used to determine Halo-tagged protein half-lives.

### Statistical Analysis

The standard error of the mean (SEM) was calculated for individual time points in each Halo-tagged protein experiment using Microsoft Excel. SEM values were then applied to their corresponding time points within the log-linear regression curves used to determine Halo-tagged protein half-lives. Results from replicate experiments were used to calculate the standard deviation, which was then divided by the square root of the number of replicates to determine the SEM. The SEM for Halo-tagged protein half-lives values were also calculated using the same formula. Half-life values were analyzed from at least 3 experiments, as previously described, and then used to calculate the SEM.

### ImageStream

1.25 10⁵ cells were collected from log-phase cultures by centrifugation at 1,200×g for 1 min. The culture media was removed, and cells were resuspended with 125 μL RPMI containing 10% FBS and HaloTag Ligand R110 (Promega Ca.) at a final concentration of 100 nM, per the company's instructions. The resuspended cells were then incubated for 90 min at 37° C. in 5% CO₂. After 90 min the cells were centrifuged at 12,000×g for 1 min and washed with PBS (2.7 mM KCl, 1.47 mM KH₂PO₄, 8.1 mM Na₂HPO₄, 137 NaCl) containing 0.1% BSA (bovine serum albumin) a total of 3 times to remove excess HaloTag Ligand R110. The cells were then resuspended in 1 mL PBS, and stained with SYTO 17 red fluorescent nucleic acid stain (Invitrogen) at a final concentration of 10 nM for 10 min, per manufacturer's instructions. The cells were washed once more, and resuspended with 200 μL PBS before being analyzed using ImageStream®^{X} Mark II Imaging Flow Cytometer (MilliporeSigma). Data analysis was done using the IDEAS 6.2's (Millipore) nuclear localization analysis Wizard.

### Confocal Imaging

1.25×10⁵ cells were collected from log-phase cultures by centrifugation at 1,200×g for 1 min. The culture media was removed, and cells were resuspended with 125 μL RPMI containing 10% FBS and HaloTag Ligand R110 (Promega Ca.) at a final concentration of 100 nM, then incubated for 90 min at 37° C. in 5% CO₂. After 90 min the HaloTag Ligand R110 labeled cells were centrifuged at 1,200×g for 1 min and washed with PBS (2.7 mM KCl, 1.47 mM KH₂PO₄, 8.1 mM Na₂HPO₄, 137 NaCl) containing 0.1% BSA (bovine serum albumin) a total of 3 times to remove excess ligand. The washed cells were then resuspended in 1 mL of PBS and stained with SYTO 17 red fluorescent nucleic acid stain (Molecular Probes, Inc. OR) according to the manufacturer's protocol. After the incubation period, the cells were centrifuged at 1,200×g for 1 min and washed once with PBS. Once resuspended in 300 μL of PBS, cells were transferred to a 12 mm glass base dish and imaged with a Leica TCS SP8 confocal imaging system (Leica Microsystems Inc, IL) using an HC PL APO 40×/1.3 oil CS2 objective. Digital images were rendered, and signal intensities were analyzed using Imaris visualization and analysis software (Bitplane Inc. MA). Cellular localization of HaloTaged proteins was determined by calculating the ratio of mean HaloTag signal intensities within the nucleus versus the cytosol. The nuclear area was established using the SYTO 17 red fluorescent nucleic acid stain, and the cytoplasmic region was determined using the diffuse EBFPII signal expressed by our lentiviral vectors.

### Results

### LMO2 Turnover is Mediated by Ubiquitin-Proteasomal System and is Inhibited by LDB1

We first approached kinetic analysis of LMO2 turnover by quantitative western blotting after cycloheximide treatment. We observed half lives in the range of 8-10 hours for endogenous LMO2 in K562, MOLT4, and LOUCY leukemia cells; the half-life of exogenous LMO2 in Jurkat cells was measured at approximately 7 hours. However, LDB1 decay was not observed by immunoblot within this same time frame. We were at the detection limits of our cycloheximide chase assay where cycloheximide toxicity is a confounding issue. Accordingly, we developed an alternative approach to analyze LMO2 and its associated proteins in live cells without metabolic perturbation and without toxins. We produced recombinant LMO2 tagged at its amino terminus with the Halo enzyme. Our prior results showed that carboxyl terminal tags on LMO2 impeded its degradation so we focused on amino terminal tagging. We expressed Halo-LMO2 in Jurkat cells, which do not express endogenous LMO2, where the recombinant protein had enhanced steady state abundance with LDB1 co-expression, implying direct binding with LDB1. This was confirmed by co-immunoprecipitation (co-IP) of Halo-LMO2 with FLAG-LDB1. Confocal microscopy showed that Halo-LMO2 was localized predominantly in the nucleus. Thus, based on all of our conventional assays, Halo-LMO2 behaved just like untagged LMO2.

In order to force expression of multiple components of the LDB1/LMO2 complex in various cell lines individually and in combination, we developed multiplexed lentiviral expression vectors allowing fluorescence-based sorting and drug selection (see FIG. 1). Then, we implemented pulse chase analysis of Halo-tagged polypeptides by standard flow cytometry. We pulsed cells with the membrane-permeable fluorochrome, R110, and analyzed cellular fluorescence and R110 decay (i.e. chase) through the FITC channel throughout our experiments. We called this technique for analyzing protein turnover, the HaloLife assay. After a 90 min pulse of R110, we plotted the decay of fluorescence for untagged Halo protein and for Halo-LMO2 in the presence or absence of bortezomib, a specific 26S proteasomal inhibitor used in proteomic analysis of ubiquitinated moieties and also currently used to treat T-ALL. Bortezomib was tested with or without co-expression of HA-LDB1 or HA-LDB1ΔLID, which cannot bind LMO2. The curves fit a typical first order exponential decay. Untagged Halo protein showed very slow protein turnover, whereas Halo-LMO2 had a t_{1/2}=6.6 hours, approximately the same t_{1/2} calculated from cycloheximide experiments. Co-expression of HA-LDB1 increased Halo-LMO2 t_{1/2} to 20.6 hours (P=1.12E-5). Similarly, bortezomib increased Halo-LMO2 t_{1/2} to 20.2 hours. In contrast, Halo-LMO2 was degraded faster with co-expression of HA-LDB1ΔLID (t_{1/2}=4.0 hours, P=1.26E-3). In summary, the presence of LDB1 markedly stabilized LMO2 as measured by the HaloLife assay. Halo-LMO2 turnover was reduced by bortezomib, implicating the ubiquitin-proteasomal pathway as the mechanism of degradation. Also, LDB1ΔLID, which is deficient in LMO2 binding but capable of homodimerization, increased the degradation of LMO2, a dominant negative effect which was previously observed in multiple leukemic cell lines.

### Specific LMO2 Lysines are Required for Stabilization and are Critical for Binding to LDB1

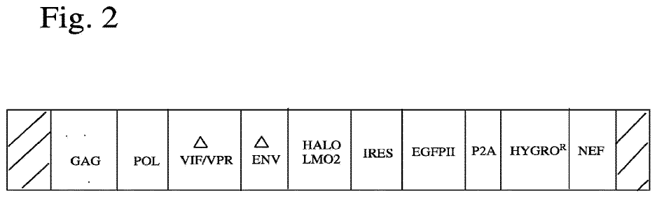

The turnover of LMO2 is particularly intriguing since it is a known driver in T-cell leukemia and an essential factor in AML. Thus, the degradation of LMO2 could be exploited therapeutically to deplete the protein in diverse leukemias and lymphomas. Our prior experiments had discovered important features about the LMO2/LDB1 interaction: (1) binding is a prerequisite for LMO2 stabilization; (2) R³²⁰LITR within LDB1 are the key interacting residues and single residue substitutions within RLITR reduce LMO2 binding to LDB1; (3) 1322 was accommodated by a hydrophobic pocket within LMO2 formed by L64 and L71. Based on these data, we applied the HaloLife assay towards assessing the turnover of various mutant LMO2 proteins. Halo-LMO2 (L64A, L71A) was significantly reduced in steady state abundance, and had faster turnover by measured t_{1/2}=1.5 h compared to t_{1/2}=6.2 h for Halo-LMO2. To identify the lysine residues within LMO2 that are potential sites for ubiquitination, we mutated the 10 lysines in the protein to arginine. Unexpectedly, lysine-less mutant LMO2 [denoted K(0)] had significantly faster turnover than LMO2 WT, t_{1/2}=4.0 h versus 6.2 h (P=1.06E-3). We discovered that LMO2 K(0) was compromised in binding LDB1 as evidenced by reduced co-immunoprecipitation. We noted there were two lysines, K74 and K78, in proximity to the LMO2 hydrophobic binding pocket interfacing with LDB1 R³²⁰LITR. Halo-LMO2 (K74R, K78R), a mutant protein with only these two key lysines mutated and the remaining 8 lysines intact, showed significantly faster turnover, measured t_{1/2}=3.9 h versus to t_{1/2} of Halo-LMO2 K(0) (P=1.76E-3). We also tested the reciprocal mutant, where we left K74 and K78 intact and mutated the remaining 8 lysines to arginine. As shown in FIG. 2B, this mutant LMO2, Halo-LMO2 K(0) (K74, K78) had a measured t_{1/2}=5.5 h, statistically insignificant (P=0.107) to the measured t_{1/2} of Halo-LMO2 WT. We then tested single substitutions at K74 and K78. Halo-LMO2 K(0) (K74) had a measured t_{1/2}=4.8 h that was significantly (P7.28E-3) reduced compared to WT Halo-LMO2 whereas Halo-LMO2 K(0) (K78)'s t_{1/2} was not significantly different, t_{1/2}=5.1 h (P=0.09). Intriguingly, K74 is conserved within all nuclear LIM-only proteins whereas K78 is unique to LMO2. Both K74 and K78 restored binding of the lysineless LMO2 to LDB1. Within lysineless proteins, the amino termini can serve as sites for ubiquitination. In order to show that the N-terminus of this version of LMO2 was critical for ubiquitin modification, we inserted a native LMO2 sequence translated from the longest transcript of the distal LMO2 promoter, creating a super-stable protein, Halo-N+LMO2 K(0) (K74, K78) measured t_{1/2}=25 h (P=4.47E-3). In summary, we identified K74 and K78 within LMO2 as essential for LDB1 binding and for normal levels of protein turnover.

Next, we examined the turnover of Halo-LMO2 in Jurkat, KOPT-K1, and K562 leukemia cells, which have various levels of LDB1 and LMO2. Jurkat cells are derived from T-ALL and express endogenous LMO1 but no LMO2; KOPT-K1 cells have a chromosomal translocation that results in overexpression of endogenous LMO2; and, K562 are aneuploid chronic myelogenous leukemia cells, resemble HSPCs, and express abundant endogenous LMO2 and LDB1. Halo-LMO2 t_{1/2} was comparable in Jurkat and K562 cells, measured at 6.2 h versus 6.4 h, respectively. The super-stable Halo-N+LMO2 K(0) (K74, K78) was similarly prolonged, t_{1/2}=25 and t_{1/2}=20.9, respectively. In contrast, Halo-LMO2 t_{1/2} measured 1.3 h in KOPT-K1 cells. The fast turnover in KOPT-K1 cells suggested to us that forced expression of Halo-LMO2 was competing with high endogenous LMO2 for the LDB1 LID. K562 cells had approximately equivalent abundance of LMO2 compared to KOPT-K1 cells, however, Halo-LMO2 turnover in K562 cells was not as fast perhaps due to the increased expression of endogenous LDB1 in comparison to KOPT-K1 cells. Competition amongst LIM domain proteins is an important determinant of neuronal cell type specificity in the spinal cord. To test this competition model and its effect upon turnover, we measured Halo-LMO2 t_{1/2} and the effects of co-expression of competing nuclear LIM domain proteins: LMO2-HA, LMO1-HA, LMO4-HA, LHX9-HA, and ISL2-HA. These HA-tagged proteins expressed at various levels in Jurkat cells but their forced co-expression increased the turnover of Halo-LMO2. These results on t_{1/2} normalized to the level of expression achieved, suggested an approximate order of affinity between LIM domain proteins for LDB1 LID. LMO2-HA was most competitive followed by LMO1, LMO4, LHX9, and ISL2. The LIM domain proteins that enhanced Halo-LMO2 turnover showed greater conservation of the key residues that we identified for LID binding, L64, L71, K74, and K78. All the LIM proteins tested had L64 conserved, however, only LMO1 and LMO2 have L71. LMO4 and LHX9 have a cysteine residue in place of K78 but have conserved K74 at the comparable position. Fitting this logic, ISL2, the protein that had no effect upon Halo-LMO2 turnover suggesting that ISL2 was the weakest competitor for LID binding, has an arginine residue in place of K74 and a threonine residue in place of K78.

We also co-expressed other known LMO2 binding partners and measured their effects on LMO2 turnover. TAL1 increased Halo-LMO2 t_{1/2} to 8.9 h (P=0.017) but LYL1 did not change it from WT levels (6.9 v. 7.0 h, P=0.75). Co-expression of Myc-GATA2 and Myc-GATA3 both significantly decreased Halo-LMO2 to 4.9 (P=0.013) and 4.8 h (P=0.011), respectively. Myc-GATA3 expressed weakly but had a substantial effect on Halo-LMO2. Finally, Halo-LMO2 had a measured t_{1/2} of 7.7 h with HA-SSBP2 co-expression, a statistically insignificant change from WT turnover.

### LDB1 is a Long-Lived Protein in Leukemia Cells

Based on the stabilization of LMO2, we suspected that LDB1 itself may be long lived, and therefore directly measured its turnover by Halo-tagging to confirm this hypothesis. Halo-LDB1 stability was consistent across diverse cell lines, measuring t_{1/2} of 23.6-27.6 h in Jurkat, KOPT-K1, and K562 cells, respectively. Halo-LDB1 turnover was inhibited by bortezomib. Prior studies had implicated K134 and K365 residues within LDB1 as affecting its degradation. Compared to LDB1 WT, which had t_{1/2} of 27.7 h, LDB1 (K134R) and LDB1 (K365R) half-lives were prolonged, t_{1/2}=77.2 h and t_{1/2}=48.2 h, respectively. Immunoblots of LDB1 showed two closely migrating bands, the slower band being enhanced in abundance with N-ethylmaleimide (NEM). This slower migrating band was not observed in blots for LDB1 (K134R) suggesting the addition of monoubiquitin at this residue.

In MEL and CHO cells, LDB1 stabilization was dependent upon Single Stranded DNA-Binding Protein 2 (SSBP2). In contrast to these studies, LDB1 abundance did not increase with forced expression of SSBP2 or SSBP3 in any of the leukemic lines analyzed. We directly tested the turnover of SSBP2 and SSBP3 by HaloLife analysis. Each paralog tested, SSBP2, SSBP3, and SSBP4, had faster turnover than LDB1, measured at t_{1/2}=5.1 h and t_{1/2}=6.8 h, and 7.6 h, respectively. SSBP2 and SSBP3 showed longer half-lives with LDB1 co-expression. SSBP2 and SSBP3 stabilization was not seen with co-expression of LDB1ΔLCCD, the interaction domain between SSBP proteins and LDB1. However, the LDB1ΔLCCD mutant protein expressed at lower steady state abundance, suggesting that there could be mutual folding and/or stabilization between SSBP proteins and LDB1.

We used the HaloLife assay to analyze the turnover of GATA factors 1-3. Each GATA factor had a turnover faster than LDB1 but with major differences. Halo-GATA1 had a t_{1/2} of 6.2 h, Halo-GATA2 had a t_{1/2} 2.0, and Halo-GATA3 had fastest turnover with t_{1/2}=1.2 h. These results generated by the HaloLIfe assay were in agreement with the half-lives observed in cycloheximide chase experiments. In summary, the HaloLife assay showed that every subunit of the LDB1/LMO2 complex had a shorter half-life than LDB1.

### TAL1 and LYL1 are Stabilized by the LMO2/LDB1 Complex

TAL1 and LYL1 are necessary cooperating drivers in LMO2-induced leukemia. These class II bHLH proteins are known binding partners of LMO2. The binding interface between TAL1 and LMO2 requires F238 within the second helix of the bHLH domain, which is conserved as F201 within helix-2 of LYL1. We tested the turnover of Halo-TAL1 and Halo-LYL1 and specific mutants containing F238 and F201, respectively, by the HaloLife assay. Halo-TAL1 had a t_{1/2} of 4.2 h and Halo-LYL1 had a t_{1/2} of 1.8 h. LMO2-HA co-expression did not significantly (t_{1/2}=5.6 h with LMO2 v. t_{1/2}=4.2 h without LMO2, P=0.215) stabilize TAL1 but stabilized LYL1(t_{1/2}=4.3 h v. 1.8 h, P=0.015). HA-LDB1 co-expression markedly stabilized Halo-TAL1 and Halo-LYL1 to t_{1/2}=19.9 h and t_{1/2}=20.5 h, respectively. This effect was only observed in the presence of LMO2. Similarly, Halo-TAL1 and Halo-LYL1 half-lives were similar to WT levels with co-expression of HA-LDB1ΔLID. Thus, LDB1's stabilization effect was not observed without LMO2 binding. To test the requirement for bHLH to LMO2 binding, we created mutant Halo proteins, Halo-TAL1(F238D), Halo-TAL1(F238G), Halo-LYL1(F201D), and LYL1(F201G), all of which were compromised in LMO2 binding in co-immunoprecipitation assays. As expected, LMO2 did not stabilize these proteins. Each mutant bHLH protein had a measured t_{1/2} comparable to its WT counterpart. HA-LDB1 co-expression increased the t_{1/2} of Halo-TAL1(F238D) to 10.7 h (P=0.014). Similarly, Halo-LYL1(F201D) was stabilized by HA-LDB1 co-expression to t_{1/2} of 3.7 h (P=0.012). Thus, aspartic acid substitutions for F238 in TAL1 and F201 in LYL1 completely abrogated LMO2-induced stabilization but partially abrogated LDB1 induced stabilization. The F238D and F201D mutants may still retain some LMO2 binding especially since LDB1 stabilizes LMO2 and increases its steady state abundance. In contrast, glycine substitutions at the same residues completely abrogated both LMO2's and LDB1's effects. In summary, Halo-TAL1 and Halo-LYL1 half-lives in Jurkat cells are partially stabilized by LMO2 co-expression. Their half-lives are markedly prolonged by LDB1 co-expression but only if the proteins have intact LMO2 binding.

### Complex Assembly and Function

Our results implied that intact binding interactions between all of the components created a stable macromolecular complex. We analyzed whether this assembly occurred in cells and whether complex assembly has a functional effect on transcription. Each component of our complex was expressed using a lentiviral vector with unique fluorescence and drug selection (FIGS. 1 and 2). We transduced components pairwise with or without FLAG-LDB1 (F-LDB1) to test abundance and binding by co-immunoprecipitation with anti-FLAG monoclonal antibody. The measured half-lives uniformly explained increased steady state abundances of Halo-tagged proteins detected by Western blot analysis. This correlation was extended to untagged or minimally tagged (i.e. single HA) proteins as well. SSBP2 was poorly expressed in Jurkat cells so SSBP3 was transduced instead; our prior experiments had shown comparable peptide counts for SSBP3 and SSBP2 by tandem mass spectrometry of purified LDB1 complexes. HA-SSBP3 was stabilized by LDB1 but not by co-expression of LMO2. Consistent with the HaloLife results, TAL1 and LYL1 were maximally stabilized by the co-expression of both LMO2 and LDB1.

Complex assembly was analyzed by anti-FLAG immunoprecipitation via F-LDB1. Jurkat cells have abundant endogenous TAL1, which was immunoprecipitated by F-LDB1 only in the presence of LMO2. Endogenous TAL1 co-IP was augmented by co-expression of SSBP3. Forced expression of LYL1 did not effectively outcompete endogenous TAL1 for LMO2/LDB1 binding whereas SSBP3 and LYL1 co-expression reduced steady state TAL1 and TAL1 co-IP. Next, we analyzed the effects of complex formation upon gene expression. We performed a pairwise comparison of RNA-seq on Jurkat cells transduced with all complex components (i.e. LMO2, LDB1, SSBP3, and TAL1 or LYL1) versus cells transduced with empty virus, generating a ranked list of differentially expressed genes. Most of the genes on this list were maximally activated or repressed by co-expression of the full complex and not by expression of partial complex components, as found for activation of ALDH1A2, CEBPE, and NKX31, and other bona fide targets.

### DISCUSSION

In this study, we describe a novel technique to analyze the turnover of the components of the leukemogenic LMO2/LDB1 protein complex, employing Halo-tagging and fluorescence-based pulse chase analysis. The assay, which we termed HaloLife, is informative in that the turnover of tagged proteins is observed in live cells. Thus, proteins are observed in their natural milieu without pharmacologic, nutritional, or mechanical disruption. This method has the added advantage of allowing the testing of the effects of various culture conditions and small molecule therapeutics upon protein turnover. The Halo tag is advantageous because it is relatively small and monomeric, approximately the mass of GFP, which has been used in similar studies. Of course, as is the case in all epitope tagging, one must verify that the tag itself does not disrupt the behavior of the protein. In the case of the proteins presented here, each one was localized to the nucleus and retained its affinity for its physiologic partners. Also, mutations that disrupted binding had the same effect upon Halo-tagged versions as the untagged proteins themselves. The pulse chase analysis showed that the Halo protein itself was very long lived (t_{1/2}>100 h). Each Halo-tagged protein had rapid turnover compared to Halo itself, such that the fusion proteins acted as “degrons” for the Halo protein. In light of the caveats noted, the t_{1/2} measured in the HaloLife assay can be viewed as an approximation of the true half-life of the native protein. However, all the measured half-lives in this study closely matched those estimated from cycloheximide chase and quantitative immunoblotting (Lurie et al., 2008) and provided an explanation for detected changes in steady state abundance. In summary, the HaloLife has the compelling advantages of being performed in live cells, in their native cellular milieu, and at steady state without cellular disruption.

HaloLife analysis of LMO2 and its binding partners revealed a hierarchy of protein turnover with LDB1 being the most stable protein. Observed half-lives in Jurkat cells in increasing order were: Halo-LYL1 (~1.8 h), Halo-TAL1 (~4.1 h), Halo-LMO2 (~6.4 h), Halo-SSBP2 (~5.1 h), Halo-SSBP3 (~6.8 h), and Halo-LDB1 (~20-24 h). Most remarkably, co-expression of LDB1 shifted the turnover of these Halo tagged subunits so that each protein partner assumed a half-life of ~20 h in the presence of excess LDB1, approximating the measured half-life of LDB1 itself. There was no reciprocal effect since none of the partner proteins prolonged the half-life of LDB1. All proteins tested were markedly stabilized by bortezomib, suggesting degradation by the ubiquitin proteasomal system. Each protein partner had to bind to LDB1 either directly or indirectly, in the case of TAL1 and LYL1, to be stabilized. Taken together, these findings suggest that the free subunits, those unbound to LDB1, are degraded more rapidly than those bound to LDB1. Furthermore, the prolonged half-life of LDB1 suggests that it is the core subunit in the assembly of the bHLH/LMO2/SSBP/LDB1 macromolecular complex, which we term the LDB1/LMO2 holocomplex. As LDB1 binds to its direct partners, SSBP proteins or LMO2, LDB1 impedes the turnover of other components of the complex so that stepwise assembly and slow turnover increase the steady state abundance of the holocomplex. Accordingly, each subunit assumes a half-life similar to that of LDB1, suggesting that the whole complex may be degraded en masse.

Two distinct lysines within LDB1, K134 and K365, have been implicated in LDB1 turnover. Both K134R and K365R mutations markedly prolonged LDB1 turnover by the HaloLife assay compared to wild type LDB1, thereby confirming the role of these lysine residues in LDB1 stability. Neither lysine is within a domain mediating subunit binding (i.e. LDB1's LCCD, residues 200-249, is responsible for SSBP binding and the LID is comprised of residues 300-330). Thus, these residues are unlikely to be occluded from ubiquitination by SSBP or LMO proteins. On the other hand, K134 is within the dimerization domain, so K134 could be masked by homodimerization. This raises the possibility of LDB1 homodimers being more stable than monomers. We discovered a slower migrating LDB1 in the presence of N-ethylmaleimide that is consistent with a monoubiquitin conjugation to K134. If we assume this residue is only accessible in unbound LDB1, then we predict that this monoubiquitinated LDB1 is monomeric.

Although the stoichiometry of the LDB1 holocomplex has not been definitively solved, our prior mass spectrometry data do suggest stable LDB1 dimers in nuclear lysates. Interestingly, this theme of accessible lysines may be extended to the turnover of LMO2 and SSBP proteins as well. Our experiments with LMO2 implicated K74 and K78 in LDB1 binding. These residues may be sites of ubiquitination and may be exposed in free LMO2 subunits but sterically hindered in LMO2 bound to LDB1. Alternatively, K74 and K78 may be subject to other post-translational modifications such as methylation or acetylation. K78 is particularly intriguing since it is unique to LMO2 and is adjacent to a hydrophobic pocket (L64 and L71) such that neutralization of the side chain amine would favor LDB1 binding by accommodating 1322. This contact interface is supported by a crystal structure of an LMO2-LID fusion protein. We co-purified SSBP3 with FLAG-LDB1 and detected a diGly motif on K35 in the mass spectrometry data, which could be a remnant of trypsinized ubiquitin, although NEDD8 and ISG13 are other possible conjugates. Nevertheless, K35, K7, and other conserved lysines are within the LUFS domain of SSBP proteins and are expected to be masked by LDB1 binding whereas free SSBP subunits should have more accessible lysine residues for modification.

In summary, free subunits of the LMO2/LDB1 complex are rapidly degraded in comparison to the slow degradation kinetics of the holocomplex. Complex assembly may proceed through binding and stabilization by masking key lysine residues in the free subunits. Recombinant full-length proteins and a structure of the holocomplex may be able to test this model. On a more general note, our studies suggest that multisubunit protein complexes may have key core subunits with enhanced stability that can be conferred upon binding subunits.

Prolonged turnover of nuclear factors and transcription factors has been suggested to be due to their association with chromatin. The subunits of the LDB1/LMO2 complex were localized to the nucleus, at least 2-fold over cytoplasm but we could not analyze whether they were chromatin-bound. The slow turnover of the LMO2/LDB1 holocomplex obviates the need to form new chromosomal loops that co-localize enhancers to core promoters during every cycle of RNA Pol II recruitment, which would be energetically unfavorable. Notably, co-expression of all complex components resulted in maximal target gene activation or repression implying that assembly of the holocomplex is what is needed to effect gene regulation.

It is important to note that the HaloLife assays were all performed in leukemic cells. The leukemia lines were of diverse lineages. Even so, one cannot rule out a general defect in the turnover of LMO2 and LDB1 in all of these lines. The work shown here required the development of novel lentiviral vectors to allow co-expression of all complex partners in the same cell. Similar analysis in normal hematopoietic cells would be challenging but is being explored since the turnover and stoichiometry of this complex in primary hematopoietic cells is of great interest and a part of our ongoing research.

Importantly, careful analysis of this protein complex turnover has major implications for regulating these major drivers of leukemia. Recent data from mouse genetics strongly supports a role for Ldb1 in Lmo2-induced leukemia. The CD2-Lmo2 transgenic mouse model develops T-ALL with long latency but with complete penetrance (Smith et al., 2014). Conditional deletion of Ldb1 in this model abrogated T-ALL onset (UPD personal observation). Thus, Ldb1 is a required Lmo2 partner in this murine model of T-ALL. This compelling result from mouse genetics coupled with the primacy of LDB1 in a protein turnover hierarchy underscore the potential for targeting the LMO2/LDB1 interface in leukemias. If LMO2 is dissociated from LDB1 then free LMO2 and TAL1 are expected to undergo rapid degradation. Supporting this idea, the co-expression of LIM domain proteins that competed for the LID (LMO1, LMO2, LMO4, and LHX9) accelerated Halo-LMO2 turnover. ISL2, which has the least similarity to LMO2 residues responsible for LID binding, did not accelerate turnover, underscoring the determinants of LID binding as a mechanism for LIM protein competition. We predict a small molecule that could bind to the LID interface would also accelerate LMO2 turnover. Of course, such an inhibitor of LMO2 binding to LDB1 would affect normal hematopoietic stem cells as well. However, there could be a therapeutic index with higher LMO2/LDB1 holocomplex-expressing cells predicted to be more sensitive to such inhibition.

Previous work implicated RNF12 as a potential E3 enzyme responsible for LDB1 and LMO2 degradation. However, in our experiments, steady state abundance of LDB1 and other subunit proteins were unchanged with forced expression of RNF12 in Jurkat cells. Thus, additional investigation is needed to characterize the degradation machinery of the LMO2 holocomplex especially in its normal or leukemic cellular contexts, which could reveal E3 enzymes or DUBs that could be therapeutically targeted. DUB enzymes are particularly amenable to small molecule inhibition since proteolytic mechanisms have been extensively studied. An shRNA knockdown screen using the HaloLife assay showed a very compelling candidate DUB, ALG13. There were other candidates identified in our screen such as OTUD7B, but ALG13 fulfilled our screening criteria and affected all subunits with no effect upon Halo protein itself. Recently, with the development of Proteolysis Targeting Chimeras (i.e. PROTACs), there is great interest in small molecules that can induce targeted degradation by recruitment of E3s to proteins of interest. Actually, one of these PROTACs is being analyzed in phase II clinical trials with similar molecules on the horizon. In contrast, bortezomib is being tested in a randomized clinical trial in T-ALL as an addition to state of the art multiagent chemotherapy. The results from our study show that bortezomib stabilizes LMO2 oncoprotein, which can potentially antagonize the effect of chemotherapies.

We claim:

1. A system comprising:
a multiplex of lentiviral vectors for modular protein expression in human hematopoietic cell lines, wherein each vector comprises
a) an HIV-1 lentiviral backbone comprising regulatory elements, wherein the regulatory elements comprise a long terminal repeat (LTR);
b) a polylinker for the insertion of a nucleic acid molecule;
c) a visible marker gene;
d) a nucleic acid molecule encoding a proteolytic cleavage site; and
e) a selectable marker gene,
wherein, the regulatory elements are operably linked to the polylinker, the visible marker gene, and the selectable marker gene,
wherein the nucleic acid molecule encoding a proteolytic cleavage site links the visible marker gene to the selectable marker gene, and
wherein each of the lentiviral vectors differ from each other by comprising a separately identifiable visible marker gene or a different selectable marker gene.

2. The system of claim 1, wherein each of the multiplex of lentiviral vectors comprises:
a unique selectable marker gene, and
a separately identifiable visible marker gene relative to the other lentiviral vectors.

3. The system of claim 2, wherein each of the multiplex of lentiviral vectors further comprises a nucleic acid molecule encoding an amino terminal epitope tag operably linked to the polylinker or the selectable or visible marker genes.

4. The system of claim 2, wherein the system comprises three or more lentiviral vectors.

5. The system of claim 4, wherein the visible marker gene is a fluorescent protein encoded by a gene selected from the group consisting of mCLOVER3, DsREDII, mAPPLE, mSCARLET, EBFPII, mTagBFPII, EYFP, mCITRINE, CERULEAN, mKATE1.3, SMurfBV+, firefly Luciferase and green fluorescent protein (EGFP).

6. The system of claim 5, wherein each of the multiplex of lentiviral vectors further comprises a nucleic acid molecule encoding an amino terminal protein fusion tag operably linked to the polylinker or the selectable or visible marker genes.

7. The system of claim 4, wherein the selectable marker gene is an antibiotic resistance gene that confers resistance against an antibiotic selected from the group consisting of puromycin, hygromycin, G418 sulfate, neomycin, phleomycin D1, and Blasticidin.

8. The system of claim 4, wherein the nucleic acid molecule encoding a proteolytic cleavage site encodes a peptide selected from the group consisting of SEQ ID NO: 24-31.

9. The system of claim 2, wherein each lentiviral vector is an H163 lentiviral expression vector.

10. The system of claim 2, wherein the visible marker gene is a fluorescent protein encoded by a gene selected from the group consisting of mCLOVER3, DsREDII, mAPPLE, mSCARLET, EBFPII, mTagBFPII, EYFP, mCITRINE, CERULEAN, mKATE1.3, SMurfBV+, firefly Luciferase and green fluorescent protein (EGFP).

11. The system of claim 10, wherein each of the multiplex of lentiviral vectors further comprises a nucleic acid molecule encoding an amino terminal epitope tag operably linked to the polylinker or the selectable or visible marker genes.

12. The system of claim 2, wherein the selectable marker gene is an antibiotic resistance gene that confers resistance against an antibiotic selected from the group consisting of puromycin, hygromycin, G418 sulfate, neomycin, phleomycin D1, and Blasticidin.

13. The system of claim 12, wherein each of the multiplex of lentiviral vectors further comprises a nucleic acid molecule encoding an amino terminal epitope tag operably linked to the polylinker or the selectable or visible marker genes.

14. The system of claim 1, wherein the multiplex of lentiviral vectors comprises 3-10 retroviral expression vectors.

15. The system of claim 1, wherein each of the multiplex of lentiviral vectors further comprises a standard internal ribosomal entry site (IRES) nucleic acid sequence located 3′ to the polylinker site and 5′ to the visible marker gene.

16. The system of claim 1, wherein each of the multiplex of lentiviral vectors further comprises a 2A family cleavage site.

17. The system of claim 1, wherein each of the multiplex of lentiviral vectors further comprises a nucleic acid molecule encoding an amino terminal epitope tag operably linked to the polylinker or the selectable or visible marker genes.

18. The system of claim 1, wherein the nucleic acid molecule encoding a proteolytic cleavage site encodes a peptide selected from the group consisting of SEQ ID NO: 24-31.

19. The system of claim 18, wherein each of the multiplex of lentiviral vectors further comprises a nucleic acid molecule encoding an amino terminal epitope tag operably linked to the polylinker or the selectable or visible marker genes.

20. The system of claim 1, wherein the human hematopoietic cell lines are selected from Jurkat, KOPT-K1, K562 leukemia cells, HEK 293T, U937, KOPT-K1, and LOUCY.
